(19) United States
(12) Patent Application Publication
(10) Pub. No.: US 2015/0290314 A1
(43) Pub. Date: Oct. 15, 2015

(54) MARKER VACCINE

(71) Applicant: Intervet Inc., Summit, NJ (US)

(72) Inventors: Hans de Smit, Boxmeer (NL); Benjamin Lamp, Vienna (AT); Hans Tillman Ruemenapf, Vienna (AT); Eveline Wentz, Vienna (AT)

(73) Assignee: Intervet Inc., Summit, NJ (US)

(21) Appl. No.: 14/423,291

(22) PCT Filed: Aug. 28, 2013
(86) PCT No.: PCT/EP2013/067771

§ 371 Date: Feb. 23, 2015

(30) Foreign Application Priority Data
Aug. 29, 2012 (EP) 12182260.5

(51) Int. Cl. A61K 39/12; A61K 45/06; G01N 33/573; C12N 7/00

(52) U.S. Cl. CPC A61K 39/12; C12N 7/00; A61K 45/06; G01N 33/573; A61K 2039/5252

(57) ABSTRACT

The present invention relates to replication-competent Bovine viral diarrhoea viruses (BVDV), Classical Swine Fever viruses (CSFV), Ovine Border Disease viruses (BDV) and atypical pestiviruses having a modification in an epitope of a viral protein, to their use as a medicament, to their use as a vaccine, to vaccines comprising such replication-competent BVDV, CSFV, atypical pestiviruses or BDV and to diagnostic tests for the detection of antibodies against such viruses and for distinguishing vaccinated animals from field infected animals

The present invention relates to replication-competent Bovine viral diarrhoea viruses (BVDV), Classical Swine Fever viruses (CSFV), Ovine Border Disease viruses (BDV) and atypical pestiviruses having a modification in an epitope of a viral protein, to their use as a medicament, to their use as a vaccine, to vaccines comprising such replication-competent BVDV, CSFV, atypical pestiviruses or BDV and to diagnostic tests for the detection of antibodies against such viruses and for distinguishing vaccinated animals from field infected animals.

The genus Pestivirus is a genus within the family Flaviviridae that comprises i.a. the Bovine viral diarrhoea virus (BVDV), the Classical Swine Fever Virus (CSFV), the Ovine Border Disease Virus (BDV) and a group of viruses known as atypical pestiviruses, such as HoBi virus and Khon Kaen virus.

BVDV, CSFV, atypical pestiviruses or BDV can induce severe diseases with marked economic losses worldwide.

Bovine viral diarrhoea virus (BVDV), a member of the pestiviruses that is the causative agent of bovine viral diarrhoea, is an economically important disease of cattle world-wide. The major economic losses caused by BVDV infections are reduced fertility, abortions and the generation of persistently infected calves, which can develop fatal “Mucosal Disease”.

CSFV causes classical swine fever; a highly contagious and sometimes fatal disease in pigs that can cause considerable economic losses.

Border disease (BD) is a congenital virus disease of sheep and goats. The most frequently seen clinical signs in sheep include barren ewes, abortions, stillbirths and the birth of small weak lambs. CSFV, BVDV, atypical pestiviruses and the Ovine Border Disease Virus are genetically and structurally closely related.

Animals can be protected i.a. against CSFV and BVDV by vaccination: conventional inactivated or modified live vaccines for the protection of pigs and cattle against e.g. CSFV and BVDV infection are known in the art and are commercially available.

The pestivirus genome consists of a single-stranded RNA of positive orientation. The RNA has a length of at least 12.3 kb and contains one large open reading frame (ORF), which is flanked by non-translated regions (NTR) at both genome ends. The pestiviral ORF is translated into one polyprotein, which is co- and post-translationally processed into at least 12 mature proteins by viral and cellular proteases.

The first protein of the pestiviral ORF is N^{pro} (N-terminal protease). N^{pro} is a non-structural autoprotease that cleaves itself off the rest of the ORF encoded polyprotein, and thereby creates its own C-terminus and also the correct N-terminus for the first structural protein in the ORF, the C (core) protein.

The C protein in the ORF is followed by the other structural proteins: E^{RNS}, E1, E2 (in that order). Together the capsid (C) protein and the three glycosylated envelope proteins (E^{RNS}, E1, E2) make up the pestiviral virion. The structural proteins are followed by the non-structural proteins (p7, NS2-NS3 and NS3, NS4A, NS4B, NS5A, and NS5B). NS3 (serine protease) and NS5 (RNA-dependant RNA polymerase activity) are directly involved in viral replication.

Studies on the replication of pestiviruses have been considerably facilitated by reverse genetics systems and the discovery of autonomously replicating subgenomic RNAs (replicons) (Behrens et al., (1998), Meyers et al., (1996^{b}), Lamp, B. (2011)).

The minimal requirements for CSFV replication were investigated, for example, by creating defective CSFV genomes lacking the gene sequences for the structural proteins. It was found that the defective CSFV genomes still replicated and could be packaged into viral particles when introduced in SK-6 cells together with helper A187-CAT RNA (Moser et al., (1999)).

An autonomously replicating defective BVDV genome, which lacks part of the Npro gene sequence as well as the genes encoding C, E^{rns}, E1, E2, p7 and NS2, had been described by Behrens et al. (1998).

At present, different approaches to deal with pestiviral infection are applied in the various countries where pestiviruses cause economic damage. The fact that these different approaches are used in parallel however causes problems, as is illustrated hereunder for BVDV. The problem is however a universal problem for all pestiviruses.

BVDV and BDV occur in all countries with a few exceptions, worldwide, where ruminants are raised.

Pestiviruses circulate in wildlife animals as well, and these thus form a reservoir from which virus can spill into domestic livestock.

The development of BVDV diagnostic tests has made it possible to detect BVDV infected herds and to trace and remove persistently infected animals.

This development, in combination with severe movement restrictions and sanitary measures has allowed the Scandinavian countries to practically eradicate BVDV from domestic livestock. However, as a consequence vaccination has now been banned in these countries.

A somewhat comparable situation occurred for CSFV in Europe: at the time CSFV was practically eradicated in the EU through vaccination, a non-vaccination policy was introduced from the 1980's onwards.

However, by far most other countries have decided, due to high cattle density, intense trade and high BVDV prevalence, to still follow the approach of vaccination.

The parallel existence of these two different approaches when dealing with BVDV infection or CSFV infection has led to the following conflicting situation: vaccinated cattle cannot easily be discriminated from field-infected cattle, because in both cases antibodies against the virus will be present. Thus it is largely unknown if BVDV-antibody-positive animals are antibody-positive due to infection (in which case they may carry the virus) or vaccination. And for this reason, i.a. Scandinavian countries will not allow importation of BVDV-antibody-positive animals and meat.

This problem can theoretically be solved through the use of so-called marker vaccines. Such vaccines lack one or more of the immunogenic viral proteins, as a result of which marker-vaccinated animals will not produce antibodies against all immunogenic viral proteins. The differences in antibody-palette between vaccinated and infected animals can be shown in diagnostic tests designed for this purpose. Such tests thus allow the discrimination between vaccinated and infected animals.

This approach has e.g. been followed for the development of a marker vaccine against CSFV. This marker vaccine is in fact a subunit vaccine based upon the CSFV E2 envelope protein. Such subunit vaccines are safe and efficacious, but a drawback lies in the fact that they may be somewhat less efficacious when compared to inactivated whole virus vaccines and modified live vaccines with respect to onset of immunity.

Thus, there is a need for vaccines that have an improved efficacy profile and are suitable as a marker vaccine.

It is an objective of the present invention to provide such improved marker vaccines.

It was now surprisingly found that such improved marker vaccines can be obtained through modification of an epitope of a helicase domain of the non-structural protein NS3.

The non-structural protein NS3 has a double-function: it has a serine protease activity and an RNA helicase activity. The primary function of the helicase of the Pestiviruses is assumed to be the unwinding of the plus and minus RNA strands of the genome after the polymerase reaction. In addition there is strong evidence put forward by Riedel et al., 2012, for the helicase to be important in the intracellular assembly of infectious virus particles.

The role and function of both enzymatic activities has been described i.a. by Tautz, N. (2000), Ming Xiao (2008), Wei Cheng (2007), Tackett, A. J. (2001), Deregt, D. (2005) and by Jian Xu (1997). The publication by Jian Xu (1997) explicitly shows how related and well conserved the NS3 region, more specifically the helicase within the NS3 protein, is between e.g. BVDV and CSFV.

The helicase of the NS3 protein has been the main target for the development of diagnostic antibody detection assays such as monoclonal antibody-based ELISA's. The reason for this is clear: the NS3 helicase is 1) very immunogenic and 2) highly conserved among pestiviruses: no or practically no mutations are found in helicase. See e.g. Collet, M. S. (1992) and Bathia, S. (2008). From a diagnostic viewpoint this has the advantage that 1) antibodies against the helicase of NS3 are easily induced in the animal and 2) due to the high conservation level of helicase an antibody detection assay against helicase will recognize e.g. all BVDV or CSFV strains.

FIG. 7 gives an overview of commercially available diagnostic tests comprising monoclonal antibodies reactive with the NS3 region.

A mutant of e.g. BVDV or CSFV, having a helicase domain with a modified epitope could well form the basis of a marker vaccine: administration of such a vaccine to an animal would induce an antibody panel that differs from that of a wild-type virus and thus vaccination could be discriminated from wild-type infection.

However, due to this very high conservation level, the helicase of NS3 would be about the least preferred region of the viral genome for allowing or making mutations for the following reason: helicase is an essential enzyme for the virus, i.e. the virus is not able to replicate without the helicase activity, i.e. it is not replication-competent. The reason for the high level of conservation of helicase is common to very many enzymes: helicase is highly dependent on its primary, secondary and tertiary structure for its action, and consequently mutations would disturb the helicase activity thereby rendering the virus non-viable. Thus, it would indeed be the least preferred region of the viral genome for making mutations.

It has now surprisingly been found that unexpectedly there are certain specific regions within the helicase domains that do allow mutations while viruses carrying such mutations are still replication-competent. Moreover these mutations could be made in epitopes of helicase domains such that these modified epitopes are no longer recognized by monoclonal antibodies reactive with the wild-type form of these epitopes.

Such viruses thus have the advantage that on the one hand they are still capable of replication and thus are suitable as a basis for live vaccines, whereas on the other hand they can be discriminated from all other BVDV, BVD, atypical pestiviruses or CSFV in the sense that they have lost, contrary to wild-type BVDV, BDV, atypical pestiviruses or CSFV, their reactivity with one or more BVDV, BVD, atypical pestiviruses or CSFV specific antibodies. Moreover they do no longer induce these antibodies in an animal.

Thus, the inventors have found that, contrary to what was expected, the helicase of the NS3 protein of BVDV, BDV, atypical pestiviruses or CSFV comprises epitopes that can be modified as a result of which they do no longer react with (or induce) antibodies against the corresponding epitope on the wild-type NS3 protein but do not cause the virus to lose its replication competence.

This invention now allows the skilled person to generate replication competent BVDV, BDV, atypical pestiviruses or CSFV mutants that can form the basis of a marker vaccine.

Thus, a first embodiment of the present application relates to a replication-competent BVDV, CSFV, atypical pestiviruses or BDV having a modification in an epitope of a viral protein as a result of which the epitope is no longer reactive with a monoclonal antibody against that epitope in a wild-type BVDV, CSFV, atypical pestiviruses or BDV, wherein the epitope is located in a helicase domain in the non-structural protein NS3.

As defined herein, a replication competent BVDV, CSFV, atypical pestiviruses or BDV is a virus that can still replicate, i.e. is capable of producing infectious progeny virus. The infectious progeny virus can be replication competent infectious progeny virus or replication defective infectious progeny virus.

Such a replication competent BVDV, CSFV, atypical pestiviruses or BDV can be a virus that comprises sufficient genetic material to be able to produce infectious progeny virus that further replicates in newly infected cells (replication competent infectious progeny virus).

It can also be a virus that lacks genetic information to the extent that it is not capable of producing infectious progeny virus that further replicates in newly infected cells but is capable, when present in a complementing cell, to produce infectious progeny virus capable of single cycle infection (replication defective infectious progeny virus). Merely as an example of the latter type of virus: a BVDV genome lacking the gene encoding the E2 or E^{rns} structural protein, if present in a complementary cell line that produces the E2 or E^{rns} protein, can lead to the production of infectious progeny BVD virus capable of a single cycle infection, i.e.: replication defective infectious progeny virus.

It will be understood that the replication rate and the amount of progeny virus may be higher or lower than that produced by wild-type virus.

As defined herein an “epitope that is no longer reactive with a monoclonal antibody reactive with said BVDV, CSFV, atypical pestiviruses or BDV in its wild-type form” is considered to be an epitope that is not reactive with such monoclonal antibody at the level of reaction that a wild-type epitope would display when reacting with such monoclonal antibodies.

The level of reaction between an epitope and a monoclonal antibody reactive with that epitope can be determined according to methods known in the art. A simple method for the determination of the reaction level between the monoclonal antibody and (an epitope of) the virus is the following standard IPMA: mutant virus and wild-type virus are both grown in parallel on susceptible cells, such as SK6 cells or MDBK cells. The cells are then fixated for 20 min. at 4° with 4% paraformaldehyde in PBS and permeabilized with 0.5% Triton-X 100. After this step, the cells are incubated with the monoclonal antibody in question, diluted to an optimal concentration in PBS with 0.1% Tween 20. A secondary HRP-conjugated goat anti-mouse IgG and 3-Amino-9-EthylCarbazole substrate solution are applied for signal detection.

A virus comprising a modification in an epitope of a helicase domain of the non-structural protein NS3 according to the invention will not react in this IPMA, i.e.: it will not give a staining reaction. The cells infected with the wild-type virus, however, will be stained.

Another, even more simple method for the determination of the reaction level between a monoclonal antibody and (an epitope of) the virus is the following standard ELISA: mutant NS3 and wild-type NS3 (or even shorter fragments of these, comprising the relevant epitope) are both expressed in an expression system such as e.g. an E. coli- or Baculovirus-based expression system. The expressed proteins are coated on the well of a microtitre plate. After this step, the wells are incubated with a monoclonal antibody against the wild-type epitope, diluted to an optimal concentration in PBS with 0.1% Tween 20. A secondary HRP-conjugated goat anti-mouse IgG and TMB substrate solution are applied for signal detection.

An NS3 construct comprising a modification in an epitope of a helicase domain of the non-structural protein NS3 according to the invention will react in this ELISA with the monoclonal antibody to a lesser extent than a wild-type NS3. And this will be reflected by a lower Optical Density (OD) value of the ELISA for the mutant NS3 than for the wild-type NS3.

Preferably, a mutant according to the invention is provided that has a modified helicase epitope that shows no substantial reaction between the monoclonal antibody and the modified epitope, i.e. the OD of the ELISA test in which the mutant is tested does not substantially exceed that of the background level. However, it may be the case that there is a weak reaction between the monoclonal antibody and the modified epitope instead of an all-or-nothing reaction.

An epitope having a reaction level of less than 80% as measured by O.D. in an ELISA test when compared to the wild-type epitope is considered no longer reactive.

As mentioned above, the NS3 protein of Pestiviruses, and more specifically the helicase region of the NS3 protein has extensively been described in the literature. There are three regions in the helicase that comprise epitopes which are reactive with antiserum raised against BVDV, CSFV, atypical pestiviruses or BDV.

The tentative position of the helicase domain depends of course on the number of amino acids preceding the helicase region. There may be a slight variation between the various members of CSFV, BVDV and BDV, even within one genus. For that reason, the tentative position of the helicase domains 1, 2 and 3 for a number of known CSFV, BVDV and BDV strains is given in table 1. FIG. 6 provides an alignment of the helicase region for these strains, allowing the skilled person to identify the helicase domains in other CSFV, BVDV and BDV strains on the basis of the consensus between the helicase sequence of such strains and the helicase sequence of the strains as given in FIG. 6.

A preferred form of this embodiment relates to a replication-competent BVDV, CSFV, atypical pestiviruses or BDV according to the invention, characterized in that the helicase domain is selected from the group consisting of helicase domain 1, 2 or 3.

The position of the NS3 protease region and the helicase domains in full-length clones for several BVDV and CSFV strains is given in table 1 below. The numbering of the polyprotein for the viruses given in the table starts with “MEL”.

TABLE 1
Position of the NS3 protease region and helicase domains in full-length
clones for several BVDV and CSFV strains. The numbering of the polyprotein
for the viruses given in the table starts with “MEL”.

| | | tentative | tentative | tentative | |
|---|---|---|---|---|---|
| | | position | position | position | Related |
| | position | helicase | helicase | helicase | Accession |
| Virus Strain | protease | domain1 | domain2 | domain3 | Number |
| CSFV | 1590-1781 | 1782-1949 | 1950-2107 | 2108-2272 | J04358.2 |
| Alfort Tuebingen | | | | | |
| (p447) | | | | | |
| BVDV-1 | 1599-1790 | 1791-1958 | 1959-2116 | 2117-2281 | U63479.1 |
| CP7 | | | | | |
| BVDV-1 | 1590-1781 | 1782-1949 | 1950-2107 | 2108-2272 | U63479.1; |
| NCP7 | | | | | deletion of 9aa in |
| | | | | | NS2 |
| BVDV-1 | 1680-1871 | 1872-2039 | 2040-2197 | 2198-2362 | NC_001461.1 |
| NADL | | | | | |
| BVDV-1 | 1590-1781 | 1782-1949 | 1950-2107 | 2108-2272 | AF091605.1 |
| Oregon C24V | | | | | |
| BVDV-2 | 1664-1855 | 1856-2023 | 2024-2181 | 2182-2346 | U18059.1 |
| 890 | | | | | |
| BDV | 1587-1778 | 1779-1946 | 1947-2104 | 2105-2269 | NC_003679.1 |
| X818 | | | | | |
| NS3, Start | 1-192 | 193-360 | 361-518 | 519-683 | NS3 has the same |
| defined as | | | | | length in all listed |
| “GPAVCKK”, | | | | | pestivirus isolates |
| end defined as | | | | | |
| “GL” | | | | | |

A more preferred form of the present invention relates to a replication-competent BVDV, CSFV, atypical pestiviruses or BDV according to the invention, characterized in that the helicase domain is a helicase domain selected from the group consisting of CSFV Alfort Tuebingen, located between amino acid position 1782 and position 2272, BVDV-1 CP7, located between amino acid position 1791 and position 2281, BVDV-1 NCP7, located between amino acid position 1782 and position 2272, BVDV-1 NADL, located between amino acid position 1872 and position 2362, BVDV-1 Oregon C24V, located between amino acid position 1782 and position 2272, BVDV-2 890, located between amino acid position 1856 and position 2346 and BDV X818, located between amino acid position 1779 and position 2269.

The Examples section provides several specific mutations in these domains that yield a replication-competent virus according to the invention, and the method for generating such replication-competent viruses is generally applicable. Thus, the skilled person who wants to make additional replication-competent viruses according to the invention in addition to the viruses disclosed in the Examples section will find ample guidance to do so below.

Basically, what is needed is at least one monoclonal antibody reactive with the helicase region of the NS3 protein.

In order to obtain monoclonal antibodies against the helicase region of the NS3 protein, it suffices to express the whole helicase region or a part of said region comprising one of the domains, or a part of a domain. The most efficient way to obtain monoclonal antibodies against an epitope of the helicase region is, to use one of the many techniques available to identify (a DNA fragment encoding) an epitope, and to just express this epitope.

At this moment, a huge variety of simple techniques is available to easily identify (a DNA fragment encoding) an epitope.

Amongst the older methods are i.a. the method described by Geysen et al (Patent Application WO 84/03564, Patent Application WO 86/06487, U.S. Pat. No. 4,833,092, Proc. Natl Acad. Sci. 81: 3998-4002 (1984), J. Imm. Meth. 102, 259-274 (1987), the so-called PEPSCAN method. This is an easy to perform, quick and well-established method for the detection of epitopes. The method is well-known to man skilled in the art. This (empirical) method is especially suitable for the detection of B-cell epitopes.

Also, given the sequence of the gene encoding any protein, computer algorithms are able to locate specific epitopes on the basis of their sequential and/or structural agreement with epitopes that are now known. The determination of these regions is based on a combination of the hydrophilicity criteria according to Hopp T. P., and Woods, K. R. (1981), and the secondary structure aspects according to Chou and Fasman ((1987) and U.S. Pat. No. 4,554,101).

Methods based upon modern methods are i.a. described by Meyer, B. and Peters, Th., (2002) and by Yingming Zhao and Chalt, B. T., (1994).

For the expression of the helicase region or a part of said region comprising one of the domains or a part of a domain, bacterial, yeast, fungal, insect and vertebrate cell expression systems are very frequently used systems. Such systems are well-known in the art and abundantly commercially available.

Further ample guidance with regard to prokaryotic and eukaryotic expression is given i.a. in recent reviews and text books on expression such as:
Trepe, K., Applied Microbiology and Biotechnology, Volume 72, Number 2 (2006), 211-222
Production of Recombinant Proteins: Novel Microbial and Eukaryotic Expression Systems, edited by Gellissen, G. Publisher: Wiley-VCH, ISBN: 3527310363 edition 2005
Expression systems, edited by Michael Dyson and Yves Durocher, Scion Publishing Ltd, ISBN 9781904842439 edition 2007.

Antibodies can conveniently be raised against epitopes as provided in the Examples section. Further antibodies against other epitopes of the helicase region can be obtained by simply expressing other or larger parts of the helicase region and using these for the induction of antibodies.

The production of monoclonal antibodies has been described extensively in the art. Monoclonal antibodies, reactive with the helicase region can be prepared by immunizing inbred mice by techniques also known for decades in the art (Kohler and Milstein, (1975)).

Methods for large-scale production of antibodies according to the invention are also known in the art. Such methods rely on the cloning of (fragments of) the genetic information encoding the protein according to the invention in a filamentous phage for phage display. Such techniques are described i.a. in review papers by Cortese, R. et al., (1994), by Clackson, T. & Wells, J. A. (1994), by Marks, J. D. et al., (1992), by Winter, G. et al., (1994) and by Little, M. et al., (1994). The phages are subsequently used to screen camelid expression libraries expressing camelid heavy chain antibodies. (Muyldermans, S. and Lauwereys, M. (1999) and Ghahroudi, M. A. et al., (1997)). Cells from the library that express the desired antibodies can be replicated and subsequently be used for large scale expression of antibodies.

The production of monoclonal antibodies specifically reactive with Pestiviruses has been described already two decades ago by Deregt (1990) and by Corapi (1990).

Even more specifically, and in direct relation to the NS3-protein, ample guidance for the production of monoclonal antibodies reactive with NS3 is i.a. given by Deregt (2005) who describes the mapping of two antigenic domains on the NS3 protein. Furthermore, several commercially available and non-commercially available ELISA tests based upon antibodies reactive with NS3 protein have been described by Bourdeau, F. (2001), Chimenzo Zoth, S. (2006), Kramps, J. A. (1999), Bathia, S. (20008) and by Makoschey, B. (2007).

So, in conclusion, a monoclonal antibody reactive with an epitope of the helicase region of the NS3 protein suffices to select viruses according to the invention having a modification in that epitope of the helicase region. The Examples section provides several examples of suitable monoclonals and the literature mentioned above provides ample guidance to develop further monoclonal antibodies reactive with the helicase region.

The Examples section also provides examples of viruses having a modification in a domain of the helicase region according to the invention. The Examples also disclose general methods for making such viruses. Therefore, the Examples section provides ample guidance to the skilled person who wants to make other viruses according to the invention, instead of using the viruses described in the Examples section.

The production/selection of Replication-competent BVDV, CSFV, atypical pestiviruses or BDV having a modification in an epitope of a helicase domain of the non-structural protein NS3 such that said epitope is no longer reactive with a monoclonal antibody reactive with said non-structural protein NS3 of BVDV, CSFV, atypical pestiviruses or BDV in its wild-type form is merely a matter of producing infectious full-length clones having a modification in the helicase region of the NS3 protein. The construction of infectious full-length clones was described already two decades ago.

Full-length infectious DNA copies have been described i.a. for BVDV (Meyers et al., J., (1996)^{b}) and for CSFV (Meyers et al., (1996) a, Moormann et al., (1996), Riedel, C. et al, PLoS Pathog. 2012; 8(3):e1002598. doi: 10.1371/journal.ppat.1002598. Epub 2012 Mar. 22).

Their availability enables scientists to perform reverse genetic engineering in order to develop attenuated strains of BVDV or CSFV.

If desired, the skilled person could even chose to avoid a site-directed mutagenesis step when making a modification in an epitope of a helicase domain of the non-structural protein NS3. In that case, a DNA fragment already comprising a modification in an epitope of a helicase domain of the non-structural protein NS3 can simply be synthesized by the experimenter or be obtained commercially. It can then be exchanged with the region of the wild-type DNA encoding that helicase epitope in a full-length cDNA clone right away using basic recombinant DNA technology.

The full length infectious clone, once made, can be transfected into a mammalian cell and the cell culture can subsequently be checked for the presence or absence of progeny virus.

Full-length clones having a lethal modification in the helicase region of the NS3 protein do not fulfil the replication competence requirement and consequently will not yield progeny virus, so this step towards replication-competent BVDV, CSFV, atypical pestiviruses or BDV according to the invention is self-selective.

The next step; the testing of the reactivity of a virus having a modification in an epitope of the helicase region of the NS3 protein with a monoclonal antibody reactive with the wild-type epitope is also a simple and straightforward one. Replication-competent BVDV, CSFV, atypical pestiviruses or BDV obtained according to the first step can be tested e.g. in a classic IPMA as described above (vide supra).

Another preferred form of this embodiment of the present invention relates to a replication-competent BVDV, CSFV, atypical pestiviruses or BDV according to the invention, wherein said epitope is no longer reactive with a monoclonal antibody selected from the group consisting of the following monoclonals: mAb BVD/C16-INT, mAb 8.12.7αNS3h, Code4 and mAb 14E7αNS3h, GL3h6 as deposited with the Collection Nationale de Cultures de Microorganismes (CNCM), Institut Pasteur, 25 Rue du Docteur Roux, F-757242 Paris Cedex 15 under the following deposit numbers: BVD/C16-INT, phase-2, Sep. 7, 2012; further shortly referred to as BVD/C16-INT (CNCM 1-4658), mAb 8.12.7αNS3h, Code4 (CNCM 1-4668) and mAb 14E7αNS3h, GL3h6 (CNCM 1-4667).

The mAb 8.12.7αNS3h, Code4 (CNCM 1-4668) was provided to Intervet International B.V. by Cornell University (“CORNELL”), as represented by the Cornell Center for Technology Enterprise and Commercialization (“CCTEC”) with offices at 395 Pine Tree Road, Suite 310, Ithaca, N.Y. 14850. Intervet International B.V. obtained the right to deposit this mAb through a license agreement with Cornell University.

Another preferred form of this embodiment relates to replication-competent BVDV, CSFV, atypical pestiviruses or BDV according to the invention wherein the modification is located in the region spanning amino acid aa193-aa683 in full-length NS; NS3 starts with the conserved amino acid sequence “GPAVCKK”.

A more preferred form of this embodiment relates to replication-competent BVDV, CSFV, atypical pestiviruses or BDV according to the invention, wherein said modification is located in the amino acid sequence ₂₂₆₂IQLAYNSHENQIPVLLPKIKNGEVTDSYENYTYLNARKLGEDVPVYVYATEGEDLAVDLL GMDW₂₃₂₅, spanning the region from amino acid 2262 to 2325 in BVDV-2 strain 890, or the comparable amino acid sequence ₂₁₈₈IQLAYNSYETQVPVLFPKIRNGEVTDTYDNYTFLNARKLGDDVPPYVYATEDEDLAVELL GLDW₂₂₅₁, spanning the region from amino acid 2188 to 2251 in CSFV strain p447.

This region binds to monoclonal antibody BVD/C16-INT. Monoclonal antibody BVD/C16-INT binds to the helicase region of the NS3 protein of all CSFV, BVDV and BDV isolates. Binding requires the presence of several domains of the helicase.

The monoclonal antibody is reactive in established ELISA systems such as direct ELISA and blocking ELISA. The monoclonal is reactive with both the full length NS3 protein and a helicase domain of NS3 when expressed in a eukaryotic expression system. The monoclonal antibody is not reactive in Western blots.

Merely as an example, replacement of the amino acid sequences above with the modified sequence IQLAYNSLETPVPVAFPKVKNGEVTDAHETYELMTCRKLEKDPPIYLYATEEED provides a replication competent virus that however is no longer recognised by the monoclonal antibody BVD/C16-INT. Such a virus fulfils the requirements of a replication-competent BVDV, CSFV, atypical pestiviruses or BDV according to the invention and is thus suitable as a virus for a marker vaccine.

Another more preferred form of this embodiment relates to replication-competent BVDV, CSFV, atypical pestiviruses or BDV according to the invention, wherein said modification is located in the amino acid sequence GQKHPIEEFIAPEVMKGEDLGSEYLDIAGLKIPVEEMKN, spanning the region from amino acid 1950-1988 in CSFV p447 or the comparable region in BVDV.

This region binds to monoclonal antibody mAb 8.12.7αNS3h, Code4, that binds to the helicase region of the NS3 protein of all CSFV, BVDV and BDV isolates. The monoclonal antibody is reactive in established ELISA systems such as direct ELISA and blocking ELISA. The mAb 8.12.7αNS3h, Code4 monoclonal is reactive with both the full length NS3 protein and a helicase domain of NS3. Moreover, it is reactive with these regions regardless if they are expressed in a prokaryotic or eukaryotic expression system. The monoclonal antibody is also reactive in Western blots.

Again, merely as an example, replacement of the amino acid sequence GQKHPIEEFIAPEVMKGEDLGSEYLDIAGLKIPVE₁₉₈₄ by GQKFTIEEVVVPEVMKGEDLADDYIEIAGLKVPKK provides a replication competent virus that however is no longer recognised by the monoclonal antibody mAb 8.12.7αNS3h, Code4 (Compensatory mutations were found at Q2108L and Y2492H).

A mutation of the region MKGE to MKLE on the other hand is lethal, i.e. no replicating progeny virus is made.

Again another more preferred form of this embodiment relates to replication-competent BVDV, CSFV, atypical pestiviruses or BDV according to the invention, wherein said modification is located in the amino acid sequence ₂₁₇₄LLISEDLPAAVKNIMA₂₁₈₉ (BVDV-1 CP7), ₂₂₃₉LLISEDLPAAVKNIMA₂₂₅₄ (BVDV-2 890) or ₂₁₆₅LLISEELPMAVKNIMA₂₁₈₀ (CSFV Alfort Tuebingen/p447).

This region binds to monoclonal antibody mAb 14E7αHNS3h, GL3h6, that binds to the helicase region of the NS3 protein of all BVDV, CSFV and BDV isolates. The monoclonal antibody is reactive in established ELISA systems such as direct ELISA and blocking ELISA. The monoclonal is reactive with both the full length NS3 protein and a helicase domain of NS3, and even with only domain 3 of helicase. Moreover, it is reactive with these regions regardless if they are expressed in a prokaryotic or eukaryotic expression system. The monoclonal antibody is also reactive in Western blots.

Again, merely as an example, replacement of the amino acid sequence LLISEDLPAAVKNIMA by LLISRDLPVVTKNIMA provides a replication competent virus that however is no longer recognised by the monoclonal antibody mAb 14E7αHNS3h, GL3h6.

As mentioned above, the virus according to the invention must be replication-competent, since otherwise it cannot be produced and therefore not be practically used, e.g. in a vaccine or for diagnostic purposes.

However this does not necessarily mean that the vaccine must replicate in the target animal in order to act as a vaccine. A virus according to the present invention inherently carries its marker-characteristics (e.g. an epitope in the helicase is no longer reactive with an antibody reactive with that epitope in a wild-type virus). Therefore, the virus functions as a marker vaccine in the target animal regardless if it replicates in the target animal or not.

Thus, another form of the present embodiment relates to replication-competent BVDV, CSFV, atypical pestiviruses or BDV according to the invention, wherein said BVDV, CSFV, atypical pestiviruses or BDV is inactivated.

Another embodiment of the present invention aims at providing marker vaccines comprising a BVDV, BDV, atypical pestiviruses or CSFV according to the invention.

Marker vaccines may be based on a whole virus according to the invention, which has been inactivated (inactivated vaccines). Such vaccines have the advantage that, due to their inactivated character, they are safe. Moreover they have the advantage over the subunit-based marker vaccines mentioned above that, since they comprise the whole virus, they trigger a better immune response. BVDV, CSFV, atypical pestiviruses and BDV can be inactivated in many ways known in the art for the inactivation of BVDV, CSFV, atypical pestiviruses or BDV. Examples of physical inactivation are UV-radiation, X-ray radiation, gamma-radiation and heating. Examples of inactivating chemicals such as β-propiolactone, glutaraldehyde, binary ethylene-imine, formaldehyde and the like, all well-known in the art, are equally applicable. It is clear that other ways of inactivating the virus are also embodied in the present invention.

Alternatively, marker vaccines according to the invention may be attenuated live vaccines, comprising a live attenuated virus according to the invention which does elicit a protective immune response in the host animal, but does not invoke the viral disease due to a mutation in its genome. Live attenuated vaccines have the advantage over inactivated vaccines that they mimic the natural infection more closely. As a consequence they provide in general a higher level of protection than their inactivated counterparts.

Existing (non-marker-) live attenuated viruses can form the starting material for making a marker vaccine according to the invention. Such live attenuated viruses have extensively been described in the art (vide infra).

Live attenuated viruses for BVD and CSF are known in the art and live attenuated virus vaccines for BVD and CSF are commercially available.

Thus, another embodiment of the present invention relates to vaccines comprising a replication-competent BVDV, CSFV, atypical pestiviruses or BDV according to the invention or an inactivated replication-competent BVDV, CSFV, atypical pestiviruses or BDV according to the invention, and a pharmaceutically acceptable carrier.

Some of the promising vaccine comprise a deletion in the N^{pro} gene and/or in the E^{rns} gene, and are preferably of a cytopathic biotype. Pestivirus vaccines on the basis of such deletions have i.a. been described in PCT-Patent Application WO 99/64604, US-Patent Application US 2004/0146854, European Patent Application EP 1104676, European Patent Application EP 1013757, European Patent Application EP 1440149, European Patent EP 1751276 and by Mayer, D., et al. (2004).

For example, in EP1161537, CSFV mutants are described from which the gene encoding E^{rns} protein has been deleted (and complemented in trans).

Risatti et al. (2007), describe CSFV mutants with substitutions in the E2 region which show an attenuated phenotype. Maurer et al. (2005) also describe CSFV E2 mutants, lacking all or part of the E2 gene which showed partial protection against lethal challenge with highly virulent CSFV. Meyers et al. (1999) describe CSFV mutants with mutations in the gene encoding the E^{rns} protein that lead to mutations. In trans complemented E^{rns} deletion mutants of CSFV were described by Widjojoatmodjo et al. (2000).

It has also been suggested to use N^{pro} deletion mutants of CSFV and BVDV as vaccine candidates. A CSFV N^{pro} mutant was disclosed already in Tratschin, J. et al. They replaced the N^{pro} gene by murine ubiquitin sequences (the mutant was called vA187-Ubi) and concluded that the proteolytic activity of N^{pro} (generation of the correct N-terminus of the C protein) is essential for viral replication, but that this activity can be replaced by the proteolytic activity of ubiquitin. It was found that the mutant was completely avirulent in pigs.

Tratschin et al. found that no viable virus was obtained when the N^{pro} gene was deleted and not replaced with another protease.

Mutants, wherein N^{pro} was replaced by murine ubiquitin, were also tested for use as a live attenuated vaccine (Mayer et al., 2004).

In further research projects, the complete BVDV-N^{pro} coding sequence was deleted, and the resulting mutant was proposed as a vaccine candidate. In EP1013757 a BVDV N^{pro} deletion mutant, based on cytopathic strain NADL, lacking the complete N^{pro} sequence is described. The resulting mutant was stated to be much less infectious in cell culture and replicated slow in comparison to its wild type counterpart. Its slow growth rate was suggested to confer an attenuated phenotype.

Also Lai et al (2000) described a BVDV N^{pro} null mutant based on the NADL strain. It was highly defective in replication and achieved a production level at least 10 times lower than the wild type virus. This mutant, due to its restricted replication capacity, may also be used as a vaccine candidate. In WO2005111201 BVDV mutants are disclosed, in which deletions were made in both the N^{pro} gene and the E^{nrs} gene. It was concluded that an N^{pro} mutation or an E^{nrs} mutation only was not sufficient to prevent infection of the foetus in pregnant heifers. Only in double mutants, based on a BVDV type 2 strain NY93, infection of the foetus in pregnant heifers could be prevented (the double mutant however was only tested against a type 2 challenge, be it with another type 2 strain, and not against a BVDV type 1 challenge).

The mutants tested lacked all but the N-terminal 4 amino acids of the N^{pro} sequence.

It was noted that the mutants growth was considerably lower than for the wild type virus. To obtain better growing viruses mutants were constructed wherein either a bovine ubiquitin gene fragment or a fragment of the bovine LC3-coding sequence replaced the major part of the N^{pro} gene.

As follows from the above, (non-marker-) live attenuated viruses of e.g. CSFV and BVDV have extensively been described in the art and for BVDV and CSFV they are even commercially available. And thus, as mentioned above, such viruses constitute a very suitable starting material for the construction of viruses according to the invention, i.e. replication-competent BVDV, CSFV, atypical pestiviruses or BDV having a modification in an epitope of a helicase domain of the non-structural protein NS3, wherein said epitope is no longer reactive with a monoclonal antibody reactive with said BVDV, CSFV, atypical pestiviruses or BDV in its wild-type form.

Such viruses do inherently behave attenuated compared to their wild-type counterparts, and they can thus be used as a basis for marker viruses in a marker vaccine.

Therefore, a preferred form of this embodiment relates to vaccines comprising a replication-competent BVDV, CSFV, atypical pestiviruses or BDV according to the invention wherein said replication-competent BVDV, CSFV, atypical pestiviruses or BDV carries an attenuating mutation in the E^{nrs} or the N^{pro} gene.

It goes without saying that such viruses would be given in the amounts and through the vaccination routes indicated by the manufacturer or as indicated in the literature.

BVDV, CSFV, atypical pestiviruses and BDV are only a few examples of the many agents causing disease in ruminants, swine and sheep/goat respectively. In practice, ruminants, swine and sheep/goat are vaccinated against a number of pathogenic viruses or micro-organisms.

Therefore it is highly attractive, both for practical and economic reasons, to combine a vaccine according to the invention for a specific animal species with an additional immunogen of a virus or micro-organism pathogenic to that animal species, or genetic information encoding an immunogen of said virus or micro-organism.

Thus, a preferred form of this embodiment relates to a vaccine according to the invention, wherein that vaccine comprises an additional immunogen of a virus or micro-organism pathogenic to the animal to be vaccinated, an antibody against said immunogen or genetic information encoding an immunogen of said virus or micro-organism. An immunogen is a compound that induces an immune response in an animal. It can e.g. be a whole virus or bacterium, or a protein or a sugar moiety of that virus or bacterium.

The most common viruses and micro-organisms that are pathogenic for ruminants are Bovine Rotavirus, epizootic Haemorrhagic Disease virus, Rift Valley Fever virus, Bovine ephemeral fever virus, Bovine Herpesvirus, Parainfluenza Type 3 virus, Bovine Paramyxovirus, Bluetongue virus, Orthobunya virus, Foot and Mouth Disease virus, Mannheimia haemolytica, Pasteurella multocida and Bovine Respiratory Syncytial Virus.

Therefore, a more preferred form of the invention relates to a vaccine according to the invention, wherein the virus or micro-organism pathogenic to ruminants is selected from the group of Bovine Rotavirus, epizootic Haemorrhagic Disease virus, Rift Valley Fever virus, Bovine ephemeral fever virus, Bovine Herpesvirus, Parainfluenza Type 3 virus, Bovine Paramyxovirus, Bluetongue virus, Orthobunya virus, Foot and Mouth Disease virus, Mannheimia haemolytica, Pasteurella multocida and Bovine Respiratory Syncytial Virus.

The most common pathogenic viruses and micro-organisms that are pathogenic for swine are Brachyspira hyodysenteriae, African Swine Fever virus, Nipah virus, Porcine Circovirus, Porcine Torque Teno virus, Pseudorabies virus, Porcine influenza virus, Porcine parvo virus, Porcine respiratory and Reproductive syndrome virus (PRRS), Porcine Epidemic Diarrhoea virus (PEDV), Foot and Mouth disease virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

Therefore, an equally more preferred form of the invention relates to a vaccine according to the invention, wherein the virus or micro-organism pathogenic to swine is selected from the group of Brachyspira hyodysenteriae, African Swine Fever virus, Nipah virus, Porcine Circovirus, Porcine Torque Teno virus, Pseudorabies virus, Porcine influenza virus, Porcine parvo virus, Porcine respiratory and Reproductive syndrome virus (PRRS), Porcine Epidemic Diarrhoea virus (PEDV), Foot and Mouth disease virus, Transmissible gastro-enteritis virus, Rotavirus, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

The most common pathogenic viruses and micro-organisms that are pathogenic for sheep/goat are Foot and Mouth disease virus, Peste des petits Ruminants, Rift Valley Fever virus, Orthobunya virus, Louping Ill, Nairobi sheep disease virus, Bluetongue virus, Caprine Arthritis Encephalitis Virus (CAEV), Ovine Herpesvirus, E. coli, Chlamydia psittaci, Clostridium perfringens, Clostridium septicum, Clostridium titani, Clostridium novyi, Clostridium chauvoei, Toxoplasma gondii, Pasteurella haemolytica and Pasteurella trehalosi.

Therefore, again an equally more preferred form of the invention relates to a vaccine according to the invention, wherein the virus or micro-organism pathogenic to sheep/goat is selected from the group of Foot and Mouth disease virus, Peste des petits Ruminants, Rift Valley Fever virus, Orthobunya virus, Louping Ill, Nairobi sheep disease virus, Bluetongue virus, Caprine Arthritis Encephalitis Virus (CAEV), Ovine Herpesvirus, E. coli, Chlamydia psittaci, Clostridium perfringens, Clostridium septicum, Clostridium titani, Clostridium novyi, Clostridium chauvoei, Toxoplasma gondii, Pasteurella haemolytica and Pasteurella trehalosi.

Vaccines in general, but especially vaccines comprising live attenuated viruses must be stored at low temperature, or they have to be in a freeze-dried form. Freeze-dried vaccines can be kept under moderate cooling conditions or even at room temperature. Often, the vaccine is mixed with stabilizers, e.g. to protect degradation-prone proteins from being degraded, to enhance the shelf-life of the vaccine, or to improve freeze-drying efficiency. Useful stabilizers are i.a. SPGA, carbohydrates e.g. sorbitol, mannitol, trehalose, starch, sucrose, dextran or glucose, proteins such as albumin or casein or degradation products thereof, and buffers, such as alkali metal phosphates.

Therefore, preferably, a vaccine according to the invention is in a freeze-dried form.

In addition, the vaccine may be suspended in a physiologically acceptable diluent. Such buffers can e.g. be sterile water, a buffer and the like.

It goes without saying, that diluents and compounds for emulsifying or stabilizing viruses are also embodied in the present invention.

A suitable amount of a virus according to the invention in a vaccine would be between 10² and 10⁸ TCID₅₀ depending on the level of attenuation of the virus used. The literature cited above and the knowledge in the art would give the skilled person ample guidance to determine the amount of virus needed. In case the vaccine strains used are based upon existing, commercially available virus strains comprising an attenuating deletion, such as a deletion in the N^{pro} gene and/or in the E^{rns} gene, the manufacturer's instructions would suffice to know how much virus should be used.

As a rule of thumb, for e.g. strains carrying a mutation in the N^{pro} and/or E^{rns} gene, an amount of 10⁵ TCID₅₀ would be a very suitable amount of virus.

Vaccines according to the invention can be administered via the known administration routes. Such routes comprise i.a. intranasal, intramuscular, intravenous, intradermal, oral and subcutaneous routes.

Still another embodiment of the invention relates to a replication-competent BVDV, CSFV, atypical pestiviruses or BDV according to the invention for use as a medicament.

Again another embodiment of the invention relates to a replication-competent BVDV, CSFV, atypical pestiviruses or BDV according to the invention for use in a vaccine.

And again another embodiment of the invention relates to a replication-competent BVDV, CSFV, atypical pestiviruses or BDV according to the invention for use in the prophylaxis of Pestivirus infection in a mammal.

A marker vaccine will in principle be used in combination with a diagnostic test. Such a diagnostic test will normally be used for testing samples collected from animals that contain antibodies (e.g. serum, plasma, saliva). It must be able to discriminate between antibodies reactive with wild-type virus and antibodies reactive with the marker virus or marker vaccine.

A diagnostic test can e.g. be based upon standard diagnostic tests known in the art such as liquid phase blocking ELISA's or sandwich ELISA's. Such tests have i.a. be described by Wensvoort G. et al., (1988), by Robiolo B. et al., (2010) and by Colijn, E. O. et al., (1997).

In a basic form such a diagnostic test may comprise the wild-type version of an epitope in a helicase domain of the non-structural protein NS3 that was modified in the virus according to the invention. Such a test could e.g. comprise wells that are coated with an epitope of a helicase domain of the non-structural protein NS3. This can easily be accomplished by expressing said epitope of a helicase domain of the non-structural protein NS3 in an expression system, followed by the coating of the wells with the protein so obtained (vide supra). It goes without saying that the expression system used should allow for expression of the epitope in or close to its native conformation, i.e. such that the epitope is recognized by antibodies raised against the wild-type virus.

Merely as an example of such a test: the test may comprise an epitope comprising the sequence LLISEDLPAAVKNIMA (a wild-type epitope, recognised by the monoclonal antibody mAb 14E7αHNS3h, GL3h6) whereas the marker virus comprises an epitope comprising the sequence LLISRDLPVVTKNIMA (the modified epitope, not recognised by the monoclonal antibody mAb 14E7αHNS3h, GL3h6.).

Animals vaccinated with the vaccine according to the invention will not raise antibodies against the wild-type epitope comprising the sequence LLISEDLPAAVKNIMA used in the diagnostic test. As a consequence, this wild-type epitope will not be blocked. If, after a washing step, the well is incubated with HRPO-conjugated mAb 14E7αHNS3h, GL3h6, this mAb will bind, which will lead to a colour reaction after the substrate, e.g. TMB is added.

An animal infected with the wild-type virus will however have raised antibodies against the wild-type epitope, so these antibodies do react with the wild-type epitope used in the diagnostic test. As a consequence, this wild-type epitope will be blocked. If, after a washing step, the well is incubated with mAb 14E7αHNS3h, GL3h6, this mAb will not bind, so no or only a limited colour reaction is seen after the substrate is added.

Thus, such a diagnostic test can be used to discriminate between animals infected with a wild-type virus and animals that were vaccinated with a virus according to the invention. Likewise, vaccinated animals and subsequently infected animals can be discriminated from merely infected animals.

It is clear that although the wild-type epitope as such can be used in a diagnostic test according to the invention, it can be convenient to use a protein comprising the complete NS3, instead of the relatively short epitope as such. Especially when the epitope is for example used for the coating of a well in a standard ELISA test, it may be more efficient to use a larger protein comprising the epitope, for the coating step.

In another form of diagnostic test, the wells can e.g. be coated with a (monoclonal or monospecific polyclonal) antibody reactive with the wild-type form of an epitope in a helicase domain of the non-structural protein NS3 that was modified in the virus according to the invention. Again merely as an example: the monoclonal antibody used for coating could e.g. be one of the deposited monoclonal antibodies: mAb 14E7αHNS3h GL3h6 for the capture NS3, whereas for detection of captured NS3 a monospecific polyclonal NS3 rabbit serum could be used.

A diagnostic test based upon this principle could e.g. comprise a well coated with that monoclonal. As a first step of that test, antibodies obtained from an animal to be tested can be pre-incubated in a tube with solubilized wild-type NS3 protein and allowed to bind to the epitopes of the helicase domain; the pre-incubation step. If the animal to be tested has been infected with a wild-type virus, the antibodies raised in the animal will bind to the NS3 protein in the tube comprising all the wild type epitopes. As a result of this, said epitope will be blocked in the pre-incubation process.

If, on the other hand, the animal to be tested has been vaccinated with a virus according to the invention, no antibodies will bind to the NS3 epitope that was modified in the vaccine virus. As a result of this, said epitope will not be blocked, and thus it will remain available for binding to the coated monoclonal antibodies reactive with said specific epitope.

If the reaction mixture from the pre-incubation well is subsequently added to the wells of the test, the epitope will bind to the mAb's coated to the wells if it's not blocked by the antibodies of the animal to be tested (i.e.: the animal is vaccinated but not infected). The captured NS3 can then in a next step be detected by for example a conjugated goat anti-bovine IgG serum. The substrate will be activated and a (color) signal can be measured.

If however all NS3 epitopes were blocked by the antibodies of the animal to be tested (i.e.: the animal has been infected with wild-type virus), the epitope will not bind to the mAb's coated to the wells. A subsequent washing step will remove all NS3 so no (color) signal will appear.

As a consequence, the binding or lack of binding of the pre-incubated NS3 to the wells is indicative for the history of the animal to be tested: vaccinated (binding and therefore a color reaction) or field-infected (no binding and therefore no color reaction).

It is also possible to use, in diagnostic tests such as e.g. the two tests described above, a modified NS3 epitope according to the invention, instead of the wild-type epitope. Viruses according to the invention that comprise that modified epitope will in many cases raise antibodies against that epitope. Again, merely as an example: such test may comprise an epitope comprising the sequence LLISRDLPVVTKNIMA (the modified epitope, not recognised by the monoclonal antibody mAb 14E7αHNS3h, GL3h6.).

Animals vaccinated with the vaccine according to the invention will raise antibodies against the sequence LLISRDLPVVTKNIMA (the modified epitope, not recognised by the monoclonal antibody mAb 14E7αHNS3h, GL3h6.). As a consequence, this epitope will be blocked. If, after a washing step, the well is incubated with mAb 14E7αHNS3h, GL3h6, this mAb will not bind, which will lead to a lack of colour reaction after the substrate is added.

An animal infected with the wild-type virus will however not have raised antibodies against the modified epitope, so no antibodies will react with the modified epitope used in the diagnostic test. As a consequence, this wild-type epitope will not be blocked. If, after a washing step, the well is incubated with a mAb directed against the modified epitope, this mAb will bind, so a colour reaction will develop after the substrate is added.

The same applies m.m. for the second test described above: in that case the pre-incubation step is done with an NS3 protein with a modified epitope instead of the wild-type epitope.

Thus, such diagnostic tests can equally be used to discriminate between animals infected with a wild-type virus and animals that were vaccinated with a virus according to the invention.

Thus, again another embodiment of the present invention relates to a diagnostic test for distinguishing mammals vaccinated with a vaccine according to the invention from mammals that have been infected with a wild-type BVDV, CSFV, atypical pestiviruses or BDV, characterized in that said diagnostic test comprises an NS3 epitope of a wild-type BVDV, CSFV, atypical pestiviruses or BDV.

Another form of this embodiment relates to a diagnostic test for distinguishing mammals vaccinated with a vaccine according to the invention from mammals that have been infected with a wild-type BVDV, CSFV, atypical pestiviruses or BDV, characterized in that said diagnostic test comprises an antibody against an NS3 epitope of a wild-type BVDV, CSFV, atypical pestiviruses or BDV.

Again another form of this embodiment relates to a diagnostic test for distinguishing mammals vaccinated with a vaccine according to the invention from mammals that have been infected with a wild-type BVDV, CSFV, atypical pestiviruses or BDV, characterized in that said diagnostic test comprises a modified NS3 epitope as described in the invention.

Still another form of this embodiment relates to a diagnostic test for distinguishing mammals vaccinated with a vaccine according to the invention from mammals that have been infected with a wild-type BVDV, CSFV, atypical pestiviruses or BDV, characterized in that said diagnostic test comprises an antibody against a modified NS3 epitope as described in the invention.

Still another embodiment of the present invention relates to the use of a diagnostic test according to the invention for distinguishing mammals vaccinated with a vaccine according to the invention from mammals that have been infected with a wild-type BVDV, CSFV, atypical pestiviruses or BDV.

### LEGEND TO THE FIGURES

FIG. 1: Code4, diluted 1:5 shows distinct binding to NS3 helicase domain 2 as well as to NS3 helicase. Each lane comprises 50 ng purified protein. Lane 1: pL200 (NS3 helicase); lane 2: pW3 NS3h-D1), lane 3: pW5(NS3h-D2), lane 4: pW1 (NS3h-D3).

FIG. 2: MAbs Code4 and 49DE reaction in indirect immunoperoxidase assay. The sign “+” positive, means that antigen could be detected by the corresponding antibody; “−” negative, means that antigen could not be detected by the corresponding antibody. For Vp1756, no binding of Code4 and DE49 could be detected whereas p447 (positive control) did effect binding with both monoclonal antibodies. An anti-E2 monoclonal antibody used as a negative control could bind to Vp1756, showing that Vp1756 is replicating comparable to the Vp447 control.

FIG. 3: FIG. 3a) Schematic view of chimeric CSFV/Non-BVDV/CSFV/BDV pestivirus constructs in NS3 D3 for transient expression. Non-BVDV/CSFV/BDV pestivirus sequence are given in black; CSFV sequence in gray; Non-BVDV/CSFV/BDV pestivirus sequence terminating amino acids are indicated. Binding of BVDV/C16-INT was detected for pW111 exclusively whereas mAb WB103 also reacted with pW109. FIG. 3b) Sequence alignment of CSFV strain Alfort and BVDV Ncp7 or Non-BVDV/CSFV/BDV pestivirus, respectively (Strider 1.4f6); consensus sequence below; asterisk: amino acid is conserved; bar: amino acid is not conserved. The full NS3 non-BVDV/CSFV/BDV pestivirus nucleic acid sequence is shown in SEQ ID No.: 1, the amino acid sequence is given in SEQ ID No.: 2.

FIG. 4: Alignment of the putative 14E7 epitope sequence (a) and mutated sequence inserted in pW95 (b), substituted amino acids underlined.

FIG. 5: Western blot of VpW95 infected cell lysate. 14E7 detects NS3 at 125 kDa in p447 CSFV Alfort, but not in VpW95 mutant. Lane1: VpW95, Lane2: Vp447, Lane3: Mock infected cells.

FIG. 6: alignment of the helicase of the NS3 region of 6 pestiviruses (Please note: non-B=non-BVDV/CSFV/BDV pestivirus)

FIG. 7: overview of commercially available diagnostic tests relying on the NS3 protein.

### EXAMPLES

### Example 1

### Monoclonal Antibodies

MAb Code4 (mAb 8.12.7αNS3h, Code4; Corapi et al. 1988) was raised against BVDV 1 “Singer”. This monoclonal antibody shows a broad reactivity with pestiviruses and recognizes an epitope within nonstructural protein 3 (NS3). Non-BVDV/CSFV/BDV pestivirus NS3 is not recognized by mAb Code4. Hybridoma cells were grown in serum-free ISF medium (Seromed). Supernatant was harvested and cleared by centrifugation. The hybridoma was obtained from E. J. Dubovi, Cornell University, Ithaca, N.Y.)

MAb 49DE was raised using the BVDV 1 “NADL”. This monoclonal antibody shows a broad reactivity with pestiviruses and recognizes an epitope within NS3 (Moenning et al., 1987; Beaudeau et al., 2000). Non-BVDV/CSFV/BDV pestivirus NS3 is not recognized by mAb 49DE. A BVD/BD diagnostic ELISA containing 49DE is commercially available through Laboratoire Service International, 69380 Lissieu, France. Hybridoma supernatant of 49DE was kindly provided by Ernst Peterhans, Institute of Virology, University of Bern, Switzerland.

MAb C16 (mAb BVD/C16-INT; Peters et al., 1986) was raised against BVDV 1, “NADL”. This monoclonal antibody shows a broad reactivity with pestiviruses and recognizes an epitope within NS3 (Edwards et al., 1991). Non-BVDV/CSFV/BDV pestivirus NS3 is not recognized by mAb C16. MAb C16 was obtained through MSD animal health.

MAb WB103 was raised against BVDV 1 “Oregon C24V” (Edwards et al., 1988; Paton et al., 1991). This monoclonal antibody shows a broad reactivity with pestiviruses and recognizes an epitope within NS3. Non-BVDV/CSFV/BDV pestivirus NS3 is not recognized by mAb MAb WB103. MAb WB103 is part of a diagnostic ELISA test (PrioCHECK, Prionics AG and was purchased from VLA Weybridge, UK.

MAb WB112 was raised against BVDV 1 “Oregon C24V” (Edwards et al., 1988; Paton et al., 1991). This monoclonal antibody shows a broad reactivity with pestiviruses including Non-BVDV/CSFV/BDV pestivirus and recognizes an epitope within NS3. MAb WB112 is part of a diagnostic ELISA test (PrioCHECK, Prionics AG and was purchased from VLA Weybridge, UK.

MAb 14E7 (mAb 14E7αNS3h, GL3h6) was raised against a bacterially expressed NS3 helicase subdomain 3 of BVDV 1 “NCP7” at the Institute of Virology, Justus-Liebig University, Giessen, Germany. This monoclonal antibody shows a broad reactivity with pestiviruses and recognizes an epitope in the C-terminal part of NS3. Non-BVDV/CSFV/BDV pestivirus NS3 is not recognized by mAb 17E7. Hybridoma cells were grown in serum-free ISF medium (Seromed).

### Cells

BHK 21 and SK-6 (Kaszas, 1972) cells were grown in Dulbecco's modified Eagle's medium (DMEM) supplemented with 10% heat-inactivated fetal calf serum (FCS). The cells were maintained at 37° C. and 5% CO₂.

### Generation of Bacterial Expressed Truncated NS3 Helicase

Truncations of BVDV NS3 helicase were generated by introducing deletions into plasmid pL200 that encodes the NS3 helicase domain of BVDV NCP7 with a C-terminal polyhistidin-tag. The helicase was divided into three domains according to the NS3 model of the related NS3 molecule from Hepatitis C Virus (HCV). A series of plasmids was constructed in which only a single poly his tagged NS3 domain was expressed (NS3 D1-his, NS3 D2-his, NS3 D3-his). Mutagenesis was performed by PCR using the primers listed in table 1 as recommended by the supplier (Pfu DNA polymerase, Promega). All constructs were confirmed by nucleotide sequencing (SeqLab).

TABLE 1
Primers and plasmids used for the generation of
bacterial expression plasmids

| Generated | | |
|---|---|---|
| plasmid | Primer | Primer sequence |
| pW1 | E1fw | 5′-CAGGAAACAGCAACCGGGTCAAAG- |
| NS3 D3-his | | 3′ |
| | CST231rev | 5′-GCTAGCCATATGTATATCTCCTTC-3′ |
| pW2 | E2fw | 5′-CACCACCACCACCACCACCATC-3′ |
| NS3 D1-his | E3rev | 5′-TGTTGTGGTTACTGACCCTGC-3′ |
| pW5 | E5fw | 5′-GGGCAAAAACACCCAATAGAAG-3′ |
| NS3 D2-his | CST231rev | 5′-GCTAGCCATATGTATATCTCCTTC-3′ |
| pW3 | E2fw | 5′-CACCACCACCACCACCACCATC-3′ |
| NS3 D1 + | E4rev | 5′-ACTTCTATAATACCTACCGGGTTTC- |
| D2-his | | 3′ |

For the construction of pW5 (coding for NS3 D1+D2-his) the intermediate plasmid pW3 was designed.

Alternatively, Non-BVDV/CSFV/BDV pestivirus substitutions for CSFV Alfort sequences and amino acid exchanges were inserted into the p1039 plasmid (Lamp, 2010). Plasmid pL282 containing the Non-BVDV/CSFV/BDV pestivirus NS3 helicase domain and N-terminal hepta-His tag was used as a donor for Non-BVDV/CSFV/BDV pestivirus sequences. A number of plasmids were used as intermediate plasmids for cloning (p1708, p1717a, p1720, p1716, p1727a, p1722, p1729 and p1372). To increase stability two plasmids named p1710 and p1711 were constructed in backbone of vector pMT/BiP (Invitrogen). P1710 contains complete CSFV Alfort NS3 in a pMT/BiP vector backbone whereas p1711 contains complete Non-BVDV/CSFV/BDV pestivirus NS3 helicase in the same pMT backbone. P1710 and p1711 were used as templates in PCR. Resulting inserts were ligated into a pet11a bacterial expression vector (Clontech). Based on these plasmids a number of constructs with Non-BVDV/CSFV/BDV pestivirus substitutions at the N-terminal stretch of NS3 helicase subdomain 2 were generated. P1763 was generated by inserting point mutations MK₁₉₈₇LE at position to plasmid p1039 with primers. The mutagenized NS3 encoding sequences were cloned into a p1039 vector via XhoI and BglII restriction sites. Resulting plasmids (p1723, p1734, p1742) were used for bacterial expression of newly generated chimeric NS3 in Rosetta pLys cells.

TABLE 2
Non-BVDV/CSFV/BDV pestivirus substitutions in CSFV
sequence on the amino acid level, numbers refer
to CSFV Alfort genome (GenBank: U90951.1).

| | Amino acids (aa) in CSFV | |
|---|---|---|
| | substituted by analogous | |
| | Non-BVDV/CSFV/BDV | Number |
| Plasmid | pestivirus codons | of aa |
| P1718 | aa2004-aa2107 | 104 |
| P1719 | aa1950-aa2003 | 54 |
| P1723 | aa2003-aa1975 | 26 |
| P1742 | aa1950-1962 | 13 |
| P1734 | aa1950-1988 | 39 |
| P1763 | MK₁₉₈₇LE | 2 |

TABLE 3
Primers and Plasmids used for generation of precursor plasmids and bacterial
expression plasmids (Please note: non-B = non-BVDV/CSFV/BDV pestivirus).

| | PCR | | |
|---|---|---|---|
| Plasmid | Template | Primer | Sequence |
| p1708 | Vector: | CST451 | 5′-CAAGAAACACCTGTCGGCTC-3′ |
| Chimeric | p1039 | CST458 | 5′-AGTGGTTGTTACTGTGCCTGCCG-3′ |
| CSFV NS3, | Insert: | CST452 | 5′-GGGCAGAAATTCACAATTGAG-3′ |
| D2 Non-B | pL282 | CST453 | 5′-TCCTCTCAAGTACCTCCCAG-3′ |
| substituted in | | | |
| pET11a vector | | | |
| p1716 | Vector: | CST462 | 5′-GCAAAGAAATTGAAGGCCAAAGGATAC- |
| | p1710 | CST463 | 3′ |
| | | | 5′-CGCCTCTACCGCCATGTTCCTG-3′ |
| | Insert: | CST464 | 5′-GCAAAAAAATTAACCACACAGGGATAC- |
| | p1711 | CST465 | 3′ |
| | | | 5′-TGTTTCCGATGCCATCTTCCTTG-3′ |
| p1717a | Vector: | CST464 | 5′-GCAAAAAAATTAACCACACAGGGATAC- |
| | p1711 | CST465 | 3′ |
| | | | 5′-TGTTTCCGATGCCATCTTCCTTG-3′ |
| | Insert: | CST462 | 5′-GCAAAGAAATTGAAGGCCAAAGGATAC- |
| | p1710 | CST463 | 3′ |
| | | | 5′-CGCCTCTACCGCCATGTTCCTG-3′ |
| p1722 | Vector: | CST471 | 5′-TTCGATGTAATCATCAGCAAGGTC-3′ |
| | p1711 | CST464 | GCAAAAAAATTAACCACACAGGGATAC-3′ |
| | Insert: | CST470 | 5′-ATTGCCGGACTGAAGATACCAGTA-3′ |
| | p1710 | pMT rev. | 5′-CTTAGAAGGCACAGTCGAGGCTG-3 ′ |
| p1729 | Vector: | CST487 | 5′-ACCCTCTAACTCTTTCTTTGGCAC-3 ′ |
| | p1711 | CST464 | 5′-GCAAAAAAATTAACCACACAGGGATAC |
| | Insert: | CST486 | 5′-AACATGCTAGTTTTTGTGCCCAC-3′ |
| | p1710 | pMT rev. | 5′-CTTAGAAGGCACAGTCGAGGCTG-3′ |
| p1727a | Vector: | CST473 | 5′-TTCAGGTACTACCACCTCCTCAATTG-3′ |
| | p1711 | CST464 | 5′-GCAAAAAAATTAACCACACAGGGATAC- |
| | | | 3′ |
| | Insert: | CST472 | 5′-GTGATGAAAGGAGAAGACTTGG-3′ |
| | p1710 | pMT rev. | 5′-CTTAGAAGGCACAGTCGAGGCTG-3′ |
| p1763 | PCR | CST497 | 5′- |
| MK₁₉₈₇LE | Template: | | GAGAATAACATGCTAGTTTTTGTGCCCAC-3′ |
| | p1723 | CST498 | 5′- |
| | | | CAGCTCCTCTACTGGTATCTTCAGTCCGGC- |
| | | | 3′ |

TABLE 4
Cloning strategies for bacterial expression plasmids
(Please note: non-B = non-BVDV/CSFV/BDV pestivirus).

| Plasmid | Refering fragments |
|---|---|
| p1710 | Insert: p1039/XhoI/BglII |
| CSFV NS3 complete | Vector: pMT-Bip-V5-His/XhoI/BglII |
| in pMT | |
| p1711 | Insert: p1708/XhoI/BglII |
| Chimeric CSFV NS3, | Vector: pMT-Bip-V5-His/XhoI/BglII |
| D2 Non-B substituted | |
| in pMT vector | |
| p1718 | Vector: p1039/XhoI/BglII |
| | Insert: 1716/XhoI/BglII |
| p1719 | Vector: p1039/XhoI/BglII |
| | Insert: p1717/XhoI/BglII |
| p1723 | Vector: p1039/XhoI/BglII |
| | Insert: p1722/XhoI/BglII |
| p1734 | Vector: p1039/XhoI/BglII |
| | Insert: p1729/XhoI/BglII |
| p1742 | Vector: p1039/XhoI/BglII |
| | Insert: 1727a/XhoI/BglII |

### Preparation of Recombinant Proteins

Recombinant his-tagged proteins were expressed in E. coli Rosetta 2 cells (Novagen). Expression was performed at 30° C. for 2 h after addition of 1 mM isopropyl-13-D-thiogalactopyranoside (IPTG, AppliChem) at an optical density of 0.8. For harvest, cells were centrifuged and resuspended in lysis buffer A (50 mM Na₂PO₄, 300 mM NaCl, pH 7.0 to 8.0) and subjected to three cycles of freezing and thawing. Ultracentrifugation at 10⁵×g for 1 h led to separation into a soluble and an insoluble fraction. Full length NS3 helicase (pL200) could be detected in the soluble fraction. In contrast individually expressed NS3 domains required solubilization using 8M urea. Proteins were purified using ion metal affinity chromatography (IMAC) with Ni²⁺ sepharose columns (HisTrap; GE Healthcare). The purity and the yield of the protein were determined in sodium dodecyl sulfate-polyacrylamide gel electrophoresis (SDS-PAGE) and confirmed in immunoblot analysis with an anti-His tag monoclonal antibody as a control. The purified proteins served as test antigens in Western blot analysis and ELISA.

### SDS-PAGE and Immunoblotting

Separation of the proteins or cell lysates respectively happened in a polyacrylamide-tricin gel system. (Schägger, 1987). Subsequent proteins were transferred on a nitrocellulose membrane (Pall Corporation). The membrane was blocked in with a 4% dried skim milk solution in PBS with 0.1% Tween 20. Chemilumenescence reagent (Western Lightning Plus ECL; Perkin-Elmer) was used for signal detection.

### Generation of Chimeric Full-Length Clones

Bacterial expression plasmids p1719, p1723, p1734 or p1742, respectively, were digested with the restriction endonucleases SalI and EcoRI and the inserts encoding NS3 were ligated into via p1372 (CSFV replicon) into p447 (CSFV full length clone), (see table 6). Plasmids were linearized using SmaI and transcribed using SP6 RNA polymerase. 2 μg of the RNA transcripts were electroporated into 5×10⁶ SK6 cells as described previously (Riedel, 2010). Electroporated cells were seeded on 96 well-plates and incubated for 2-3 days. Virus replication was assessed by indirect peroxidase monolayer assay (IPMA) using a E2 specific monoclonal antibody (A18). Supernatants of CSFV positive cells were used for infection of new SK-6 cells to further propagate virus to allow testing for reactivity with mAbs Code4 and 49DE. For construction of pW95, in a first step mutations were introduced into p989 (nt 4440-8340 inserted in a pET-11a vector) resulting in pW94. In a second step the insert encoding NS3 was cloned via EcoRI and NgoMIV into the full-length clone p447 giving rise to full-length clone pW95.

TABLE 5
Summary of Non-BVDV/CSFV/BDV pestivirus amino acid sequences
substituted in CSFV full-length clone p447

| | Inserted Non- | |
|---|---|---|
| | BVDV/CSFV/BDV | Number |
| Plasmid | pestivirus aa sequence | of aa |
| p1721 | aa1950-aa2003 | 54 |
| p1725 | aa1950-aa1975 | 26 |
| p1744 | aa1950-aa1962 | 13 |
| p1740 | aa1950-aa1988 | 39 |
| p1756 | aa1950-aa1975, | 28 |
| | Q₂₁₀₈L, Y₂₄₉₂H | |
| p1751 | aa1950-aa1975, | 27 |
| | Q₂₁₀₈L | |
| p1752 | aa1950-aa1975, | 27 |
| | Y₂₄₉₂H | |
| pW95 | EE₂₁₇₀RD, AAV₂₁₇₅VV | 5 |

TABLE 6
Cloning strategies for bacterial expression plasmids

| Plasmid | Refering fragments |
|---|---|
| p1721 | Vector: p447/EcoRI/NgoMIV |
| | Insert: p1720/EcoRI/NgoMIV |
| p1720 | Vector: p1372/SalI/EcoRI |
| | Insert: p1719/SalI/EcoRI/Xho |
| p1725 | Vector: p447/EcoRI/NgoMIV |
| | Insert: p1724/EcoRI/NgoMIV |
| p1724 | Vector: p1372/SalI/EcoRI |
| | Insert: p1723/SalI/EcoRI/Xho |
| p1744 | Vector: p447/EcoRI/NgoMIV |
| | Insert: p1743/EcoRI/NgoMIV |
| p1743 | Vector: p1372/SalI/EcoRI |
| | Insert: p1742/SalI/EcoRI/Xho |
| p1740 | Vector: p447/EcoRI/NgoMIV |
| | Insert: p1739/EcoRI/NgoMIV |
| p1739 | Vector: p1372/SalI/EcoRI |
| | Insert: p1734/SalI/EcoRI/Xho |
| p1756 | Vector: p447/EcoRI/NgoMIV |
| | Insert: p1755/EcoRI/NgoMIV/SacII |
| p1750 | Vector: p1746/SalI/EcoRI |
| | Insert: p1723/SalI/EcoRI |
| p1751 | Vector: p447/EcoRI/NgoMIV |
| | Insert: 1749/EcoRI/NgoMIV/SacII |
| p1752 | Vector: p447/EcoRI/NgoMIV |
| | Insert: 1750/EcoRI/NgoMIV/SacII |
| p1749 | Vector: p1745/SalI/EcoRI |
| | Insert: p1723/SalI/EcoRI/Xho |

TABLE 7
Primers and plasmids used for the generation of
full-length clones

| | PCR | | |
|---|---|---|---|
| Plas- | tem- | | |
| mid | plate | Primer | Primer sequence |
| p1755 | P1750 | CST489 | 5′-CTAGAAACACCTGTCGGCTCTAAAG-3′ |
| | | CST490 | 5′-ACTCCTGTAGTATCTTCCAGGCTTC-3′ |
| p1745 | P989 | CST489 | 5′-CTAGAAACACCTGTCGGCTCTAAAG-3′ |
| | | CST490 | 5′-ACTCCTGTAGTATCTTCCAGGCTTC-3′ |
| p1746 | P989 | CST491 | 5′-CACAATAATCTGTCCAAAATAGTTGAA |
| | | | C-3′ |
| | | CST492 | 5′-GTTCCAGCTCTTGTATGTATAAGTC-3′ |
| pW94 | p989 | CST482 | 5′-CAGATCTCGTGATATCAACAGGTTG-3′ |
| | | CST483 | 5′-CCGGTGGTAACAAAAAATATAATGGC |
| | | | C-3′ |

### RNA Isolation

To assess potential reversions of the introduced mutations in viable CSFV after transfection virus RNA was prepared from infected cells using RNeasy kits (Quiagen). The purified RNA was reverse transcribed using Superscript reverse transcriptase 2 (Invitrogen) and CSFV-specific primers lead to three cDNA fragments covering NS3. Subsequently, fragments were cloned into plasmids and sequenced. If a mutation could be found in the fragment, the corresponding mutations were inserted into the original full-length clone and the virus was checked for growth in cell culture and in IPMA as described.

### Indirect Immunoperoxidase Assay

SK6 and BHK cells were fixed for 20 min at 4° with 4% paraformaldehyde in PBS and permeabilized with 0.5% Triton-X 100. After fixation, cells were incubated with the monoclonal antibody in question, diluted to an optimal concentration in PBS with 0.1% Tween 20. A secondary HRP-conjugated goat anti-mouse IgG and 3-Amino-9-EthylCarbazole (AEC, Sigma Aldrich) substrate solution were applied for signal detection.

Generation of Chimeric CSFV/Non-BVDV/CSFV/BDV Pestivirus pCite Plasmids

Epitopes for mAbs WB103 WB112 and C16 were difficult to map because these antibodies were neither reactive in Western blot analysis nor in ELISA using bacterially expressed proteins. Therefore transient eucaryotic expression of NS3 derivatives was employed. For this purpose chimeric CSFV/Non-BVDV/CSFV/BDV pestivirus NS3 helicase genes were cloned into the pCite 2a(+) vector. This vector contains a T7 promoter and an internal ribosomal entry site (IRES) that allows efficient cytoplasmic protein expression in conjunction with recombinant vaccinia virus MVA T7 that expresses T7 RNA polymerase. Based on the CSFV NS3 helicase containing pCite plasmid pL270, each NS3 helicase subdomain (D1, D2, D3) was replaced by the analogous domain of the Bugowannah virus NS3. As described above, pL282 served as a donor for Non-BVDV/CSFV/BDV pestivirus NS3 helicase sequences. pW91 (containing NS3 with domain D3 of Non-BVDV/CSFV/BDV pestivirus) and pW92 (containing NS3 with domain D1 of Non-BVDV/CSFV/BDV pestivirus) were constructed by PCR based cloning. In case of pW93, NS3 was amplified from an already existing plasmid (p1708) coding for a NS3 whereas D1 and D3 originate from CSFV and domain D2 originates from Non-BVDV/CSFV/BDV pestivirus.

Additionally, NS3 helicase containing plasmids with a chimeric D3 were engineered (pW109, pW110 and pW111) based on pL270 and pW91. In case of pW119, the N-terminal half of D3 was replaced by Non-BVDV/CSFV/BDV pestivirus (83aa). In pW110, the remaining 82aa in the C-terminal end of NS3h SD3 were replaced by Non-BVDV/CSFV/BDV pestivirus sequence. pW111 is a plasmid where only the last 38aa of D3 were substituted.

TABLE 8
Primers and plasmids used for the construction of
chimeric pCite clones; inserted cleavage sites underlined
(Please note: non-B = non-BVDV/CSFV/BDV pestivirus)

| Generated | PCR | | |
|---|---|---|---|
| plasmid | templates | Primers | Primer sequence |
| pW91 | Vector: | CST500 | 5′-GCTCCTGTAGTATCTTCCAGGCTTC-3′ |
| NS3 D3 | pL270 | CST451 | 5′-CAAGAAACACCTGTCGGCTC-3′ |
| Non-B | Insert: pL282 | CST501 | 5′-CCTGAAAACACTGCAGGTGAAAAGG-3′ |
| | | pet11a | 5′-GGTTATGCTAGTTATTGCTCAG-3′ |
| | | rev. | |
| pW92 | Vector: | CST502 | 5′-CACTGGGCAGAAACACCCTATAGAG-3′ |
| NS3 D1 | pL270 | CST458 | 5′-AGTGGTTGTTACTGTGCCTGCCG-3′ |
| Non-B | Insert: pL282 | CST503 | 5′-GTGCTCACAGTCCCGGATG-3′ |
| | | pet11afw | 5′-GGAATTGTGAGCGGATAAC-3′ |
| p1708 | Vector: | CST451 | 5′-CAAGAAACACCTGTCGGCTC-3′ |
| NS3 D2 | p1039 | CST458 | 5′-AGTGGTTGTTACTGTGCCTGCCG-3′ |
| Non-B in | Insert: pL282 | CST452 | 5′-GGGCAGAAATTCACAATTGAG-3′ |
| pet11a | | CST453 | 5′-TCCTCTCAAGTACCTCCCAG-3′ |
| pW93 | Insert: p1708 | CST515 | 5′-AAACATATGAGTGGGATACAAACGG-3/ |
| NS3 D2 | | | NdeI |
| Non-B | | CST516 | 5′-AAACTCGAGTTATAGACCAACCACCTG-3/ |
| | | | XhoI |
| | Vector: pL270 digested with XhoI/NdeI | | |
| pW109 | Vector: | CST528 | 5′-CAATTGTATAGGTTCGGGATG-3′ |
| | PW91 | CST386 | 5′-TAACTCGAGCACCACCACCACCAC-3′/XhoI |
| | Insert: pL270 | CST529 | 5′-GCGTATAACAGCTACGAGACAC-3′ |
| | | E21 | 5′-GTGGTGGTGGTGGTGCTCGAGTTA-3′/XhoI |
| pW110 | Vector: | CST527 | 5′-GAGTTGAATTGGTTCTGGGTG-3′ |
| | pL270 | CST386 | 5′-TAACTCGAGCACCACCACCACCAC-3′/XhoI |
| | Insert: pW91 | CST526 | 5′-GCTTACAATAGTTTAGAAACCCC-3′ |
| | | E21 | 5′-GTGGTGGTGGTGGTGCTCGAGTTA-3′/XhoI |
| pW111 | Vector: | CST102 | 5′-CACGTAGGGGGGTACGTCATCTCC-3′ |
| | pL270 | CST386 | 5′-TAACTCGAGCACCACCACCACCAC-3′/XhoI |
| | Insert: pW91 | CST530 | 5′-TATGCAACAGAAGAAGAAGATCTCG-3′ |
| | | E21 | 5′-GTGGTGGTGGTGGTGCTCGAGTTA-3′/XhoI |

Generation of Deleted and Truncated pCite Plasmids

Plasmids in which individual domains were deleted were prepared on the basis of pL270 and resulted in pW106 (NS3AD1), pW107 (NS3AD3) and pW108 (NS3AD2). A collection of plasmids (pW100, pW101, pW102, pW103 and pW104) represent NS3 genes with c-terminal truncations of D3. PL105 is a pCite based plasmid in which only D3 is expressed.

TABLE 9
Primers and plasmids used for the construction of truncated pCite plasmids

| Generated | PCR | | |
|---|---|---|---|
| plasmid | template | Primers | Primer sequence |
| pW100 | pL95 | CST386 | 5′-TAACTCGAGCACCACCACCACCAC-3′/XhoI |
| | | CST101 | 5′-CTGCCAGCTTCCACGGTGCC-3′ |
| pW101 | pL95 | CST386 | 5′-TAACTCGAGCACCACCACCACCAC-3′/XhoI |
| | | CST102 | 5′-CACGTAGGGGGGTACGTCATCTCC-3′ |
| pW102 | pL95 | CST386 | 5′-TAACTCGAGCACCACCACCACCAC-3′/XhoI |
| | | CST103 | 5′-TCTTATTTTTGGGAATAATACC-3′ |
| pW103 | pL95 | CST386 | 5′-TAACTCGAGCACCACCACCACCAC-3′/XhoI |
| | | CST104 | 5′-CTGCCATCGGCAGCTCTTCTG-3′ |
| pW104 | pL95 | CST386 | 5′-TAACTCGAGCACCACCACCACCAC-3′/XhoI |
| | | CST105 | 5′-CGTAGTTCATCTCTCTGAAGG-3′ |
| pW105 | pL95 | Core27l | 5′-CAAGAAACACCTGTCGGCTC-3′ |
| | | CST105 | 5′-CGTAGTTCATCTCTCTGAAGG-3′ |
| pW106 | pL270 | CST397 | 5′-ATGGGTGGTGGCCATGGTATTATCATC-3′ |
| | | CST502 | 5′-CACTGGGCAGAAACACCCTATAGAG-3′ |
| pW107 | pL270 | CST386 | 5′-TAACTCGAGCACCACCACCACCAC-3′/XhoI |
| | | CST480 | 5′-ACTCCTGTAGTATCTTCCAGGCTTC-3′ |
| pW108 | pL270 | CST525 | 5′-AGTGGTTGTTACTGTGCCTGCC-3′ |
| | | Core271 | 5′-CAAGAAACACCTGTCGGCTC-3′ |

Transient Expression of pCite Plasmids

A confluent monolayer of BHK cells was infected with vaccinia MVA T7 at a multiplicity of infection of 100 for two hours in order to allow production of T7 RNA polymerase. Then, cells were transfected with the described chimeric, truncated and subdomain-deleted pCite based plasmids using Superfect (Quiagen) according to manufacturer's instructions. All previously were used in vaccinia transfection assay. The plasmids pL270 (NS3 helicase), pL95 (full length NS3), pL261 (NS3 protease) served as controls. Immunoperoxidase assay was performed as described above.

### Results

Epitope Mapping for mAbs Code4/49DE

### Binding Properties to Bacterial Expressed Proteins

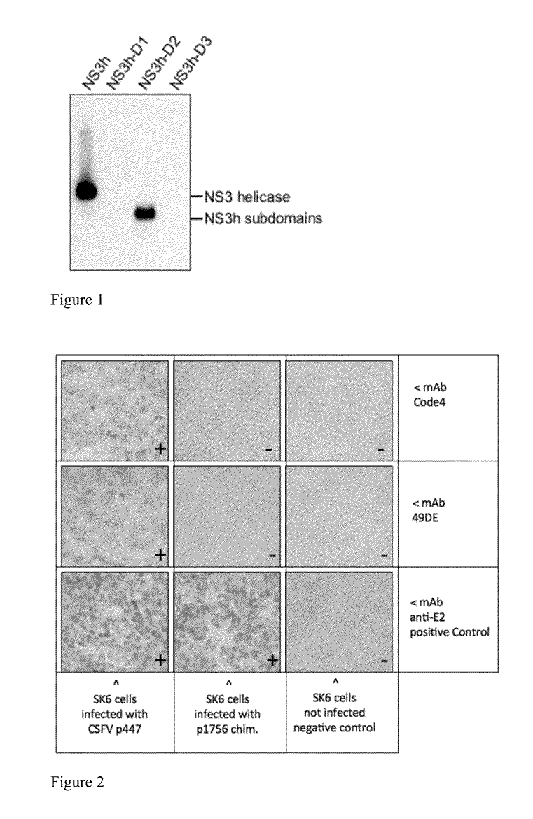

Code4 and 49DE both work well in Western blot and were tested with bacterially expressed NS3 helicase single subdomains and with full length NS3 helicase as a control. Both monoclonals showed distinct binding to NS3 helicase subdomain 2 (Code4 shown in FIG. 1). The binding of Code4 and 4DE against NS3 helicase D2 was confirmed by ELISA.

In Western blot with bacterially expressed chimeric CSFV/Non-BVDV/CSFV/BDV pestivirus NS3 helicase, 49DE and Code4 showed similar binding patterns. As expected, no binding could be detected when NS3 D2 was replaced by Non-BVDV/CSFV/BDV pestivirus sequence. Substitution of the N-terminal third of NS3 D2 (p1719; aa1950-2003) did inhibit binding of both monoclonal antibodies. In further experiments the main body of the epitope could be narrowed down to a region spanning between aa 1950-1975 of CSFV NS3. MAb 49DE did not show reactivity with an NS3 that carried amino acids 1950-1975 form Non-BVDV/CSFV/BDV pestivirus. There is evidence that the epitope of Code4 (and 49DE) likely contains amino acid 1987 and 1988 as the mutation MK₁₉₈₇LE in p1763 led to a marked binding reduction.

TABLE 10
Summary of results from immunoblotting with chimeric
NS3 helicase antigen; “−”: no binding has
been detected; “+”: binding of monoclonal
antibody has been detected; “+/−”: considerable signal reduction.

| | Amino acids (aa) in | | | |
|---|---|---|---|---|
| | CSFV substituted for | | | |
| | Non-BVDV/CSFV/BDV | Number | reaction in immune blot | |
| Plasmid | pestivirus | of aa | Code4 | 49DE |
| P1718 | aa2004-aa2107 | 104 | − | − |
| P1719 | aa1950-aa2003 | 54 | − | − |
| P1723 | aa1950-aa1975 | 26 | +/− | − |
| P1742 | aa1950-aa1962 | 13 | + | + |
| P1734 | aa1950-aa1988 | 39 | − | − |
| P1763 | MK₁₉₈₇LE | 2 | +/− | +/− |

### Recognition of Mutated Epitopes in Recombinant Viruses

Because the prime goal of this study was to generate a viable virus that inhibits binding of selected monoclonal antibodies, chimeric sequences that avoid Code4 binding with bacterially expressed antigen were cloned into a full-length p447 CSFV clone. Most of the chimeric viruses with inserted Non-BVDV/CSFV/BDV pestivirus sequences were not viable. One clone (p1725) required 2-3 days after electroporation to produce virus offspring. Sequencing of rescued virus Vp1725 revealed that positions Q₂₁₀₈L and Y₂₄₉₂H were changed. The functional importance of these rescue mutations was shown with construction of p1756. Table 11 gives a summary of constructed full-length clones and their characteristics in cell culture.

TABLE 11
Summary of constructed full-length clones for the epitope mapping
of Code4 and 49DE, characteristic properties in cell culture

| | Amino acids | | | Viable | |
|---|---|---|---|---|---|
| | (aa) in CSFV | | Growth in | clones | |
| | substituted for | Num- | cell culture | after | Compen- |
| Plas- | Non-BVDV/CSFV/ | ber | after electro- | rever- | satory |
| mid | BDV pestivirus | of aa | poration | sion | mutations |
| p1721 | aa1950-aa2003 | 54 | No | No | No |
| p1725 | aa1950-aa1975 | 26 | No | Yes | Q₂₁₀₈L, |
| | | | | | Y₂₄₉₂H |
| p1744 | aa1950-aa1962 | 13 | Yes | No | No |
| p1740 | aa1950-aa1988 | 39 | No | No | No |
| p1756 | aa1950-aa1975, | 28 | Yes | No | No |
| | Q₂₁₀₈L, Y₂₄₉₂H | | | | |
| p1751 | aa1950-aa1975, | 27 | No | Yes | Y₂₄₉₂H |
| | Q₂₁₀₈L | | | | |
| p1752 | aa1950-aa1975, | 27 | No | Yes | Q₂₁₀₈L |
| | Y₂₄₉₂H | | | | |

Only full-length clones that replicated in cell culture could be tested for the binding of mAbs Code4 and 49DE. This includes p1744 and the revertant p1756. Full-length clone p1756 is not recognized neither by mAb Code4 nor mAb 49 DE in IPMA nsd Western blot, leading to the conclusion that the epitopes of these monoclonal antibodies are identical or are located around the same area of the NS3 molecule. The reactivity of mAbs Code4 and 49DE are summarized in FIG. 2 and in table 12.

TABLE 12
Reactivity of mAbs Code4 and 49DE in indirect immunoperoxidase
assay; “−”: no binding has been detected;
“+”: binding of monoclonal antibody has
been detected; “+/−”: considerable signal reduction.

| Amino acids (aa) in | |
|---|---|
| CSFV substituted for | Reaction in |
| Non-BVDV/CSFV/BDV | immunoperoxidase assay |

| Virus | pestivirus | Code4 | 49DE |
|---|---|---|---|
| Vp1744 | aa1950-aa1962 | +/− | − |
| Vp1756 | aa1950-aa1975, | − | − |
| | Q₂₁₀₈L, Y₂₄₉₂H | | |

Vp1751 and Vp1752 were constructed in order to confirm the compensatory mutations in p1756. Each full-length clone holds the Vp1725 sequence plus one compensatory mutation from Vp1756 (Q₂₁₀₈L in Vp1751 and Y₂₄₉₂H in Vp1752). Both viruses grow well in cell culture after a 2-3 days and had established the missing compensatory mutation identical to that present in p1756.

Epitope Mapping for mAbs C16/WB103

MAbs C16 and WB103 did not show any reactivity in Western blot or in ELISA with bacterial expressed antigens. Furthermore no binding to lysate of CSFV or BVDV infected cells could be detected in Western blot analysis, indicating that C16 and WB103 recognize discontinous epitopes, possibly with a postranslational modification. Consequently, a Vaccinia MVA T7 virus based transient eucaryotic expression was established as reporter system.

Binding Properties of mAbs to MVA T7 Transient Expressed Proteins

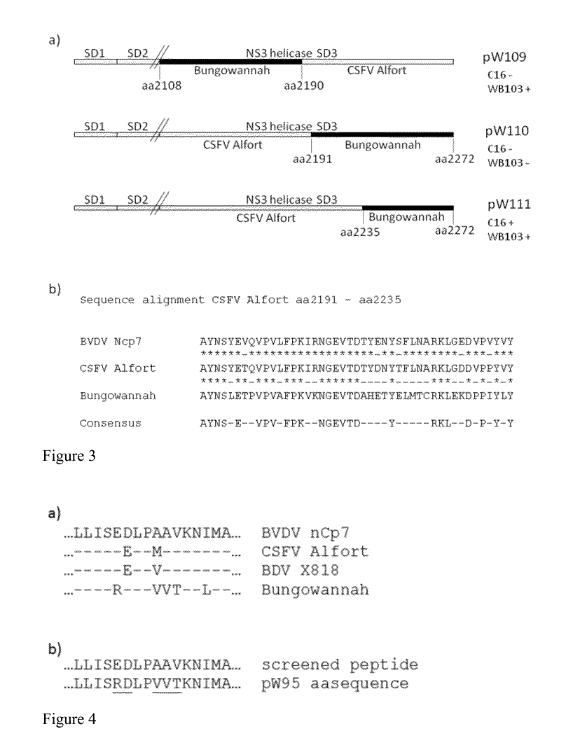

Indirect immunoperoxidase assay was performed on a monolayer of vaccinia T7 transient BHK cells transfected with various pCite derived plasmids in order to map mAbs C16 and WB103. Both mAbs, C16 and WB103, clearly bound to NS3 helicase domain whereas no binding to the protease domain could be detected. Substitutions of CSFV sequences by Non-BVDV/CSFV/BDV pestivirus revealed that mAbs C16 and WB112 both bind to domain 3 of NS3. When D3 was truncated C-terminally, binding of both mAbs was aborted when aa2235-2272 or a larger stretch of aa were removed (aa2272 represents the C-terminal end of NS3 D3). Hence, D3 was split into two parts, whereas either the N-terminal end (pW109, aa2108-2207) or the C-terminal end (pW110, aa2208-2272) represented Non-BVDV/CSFV/BDV pestivirus sequences. Additionally, a plasmid with a smaller Non-BVDV/CSFV/BDV pestivirus segment was prepared (pW111, aa2235-aa2272) (FIG. 3).

Differences in the binding affinity of mAbs C16 and WB103 could be observed with pW109. MAb C16 failed whereas mAb WB103 clearly recognized pW109 transfected cells. Therefore, the main body of the epitope of WB103 locates between aa 2207 and aa 2265. The epitope of mAb C16 likely includes amino acids N-terminal of aa 2207.

MAb C16 as well as mAb WB103 neither bound to individually expressed NS3 D3 nor to constructs where D3 was expressed in context with D1 or D2. Nevertheless, experiments with chimeric NS3 helicase clearly indicate binding to NS3D3. Hence it is supposed that C16 and WB103 bind to sensitive structural epitopes that are unable to fold correctly except in a full-length NS3 helicase consisting of all three subdomains.

TABLE 13
Binding of C16 and WB103, respectively, to transient expressed
NS3 variants. “+”: positive signal, binding of mAb
was detected; “−”: negative signal, no binding detected.

| Plasmid | Characteristics | C16 | WB103 |
|---|---|---|---|
| pL95 | Full length CSFV | + | + |
| | NS3 | | |
| pL261 | CSFV NS3 protese | − | − |
| pL270 | CSFV NS3 helicase | + | + |
| pW91 | NS3 D3 substituted by | − | − |
| | Non- | | |
| | BVDV/CSFV/BDV | | |
| | pestivirus | | |
| pW92 | NS3 D1 substituted by | + | + |
| | Non- | | |
| | BVDV/CSFV/BDV | | |
| | pestivirus | | |
| pW93 | NS3 D2 substituted by | + | + |
| | Non- | | |
| | BVDV/CSFV/BDV | | |
| | pestivirus | | |
| pW109 | aa 2108-2190 | − | + |
| | substituted by Non- | | |
| | BVDV/CSFV/BDV | | |
| | pestivirus | | |
| pW110 | aa 2191-2272 | − | − |
| | substituted by Non- | | |
| | BVDV/CSFV/BDV | | |
| | pestivirus | | |
| pW111 | aa 2235-2272 | + | + |
| | substituted by Non- | | |
| | BVDV/CSFV/BDV | | |
| | pestivirus | | |
| pW100 | aa 2265-2272 deleted | + | + |
| pW101 | aa 2235-2272 deleted | − | − |
| pW102 | aa2208-2272 deleted | − | − |
| pW103 | aa2175-2272 deleted | − | − |
| pW104 | aa2145-2272 deleted | − | − |
| pW105 | D3 indiv. expressed | − | − |
| pW106 | NS3 D1 deleted | − | − |
| pW107 | NS3 D3 deleted | − | − |
| pW108 | NS3 D2 deleted | − | − |

### Epitope Mapping for mAb WB112

As for mAbs C16 and WB103, mAb WB112 did not react with bacterially expressed proteins in Western blot or in ELISA. Therefore, a transient eucaryotic expression system was used.

### Binding Properties to Transiently Expressed Chimeric CSFV/Non-BVDV/CSFV/BDV Pestivirus NS3 Helicase

MAb WB112 was tested in a eucaryotic expression system using vaccinia infected, BHK cells transfected with the plasmid construct listed in Table 14. MAb WB112 recognizes NS3 within the helicase domain and is crossreactive with swapped domains of Non-BVDV/CSFV/BDV pestivirus NS3. Using NS3 constructs that lack individual domains, binding was abrogated if D2 was deleted. Very likely the epitope of mAb WB112 is located within D2 of NS3.

TABLE 14
Binding of mAb WB112 to transiently expressed NS3 variants.
“+”: positive signal, binding of mAb was detected;
“−”: negative signal, no binding detected.

| Plasmid | Characteristics | WB112 |
|---|---|---|
| pL95 | Full length CSFV | + |
| | NS3 | |
| pL261 | CSFV NS3 protease | − |
| pL270 | CSFV NS3 helicase | + |
| pW91 | NS3 D3 substituted by | + |
| | Non- | |
| | BVDV/CSFV/BDV | |
| | pestivirus | |
| pW92 | NS3 D1 substituted by | + |
| | Non- | |
| | BVDV/CSFV/BDV | |
| | pestivirus | |
| pW93 | NS3 D2 substituted by | + |
| | Non- | |
| | BVDV/CSFV/BDV | |
| | pestivirus | |
| pW106 | NS3 D1 deleted | − |
| pW107 | NS3 D3 deleted | + |
| pW108 | NS3 D2 deleted | + |

### Epitope Mapping for mAb 14E7

### Binding Properties to Bacterial Expressed Proteins

14E7 was established by immunizing mice with bacterially expressed NS3. MAb 14E7 is reactive with several pestiviruses in Western blot, ELISA and IPMA but not with Non-BVDV/CSFV/BDV pestivirus.

Mapping mAb 14E7 using truncated NS3 D3

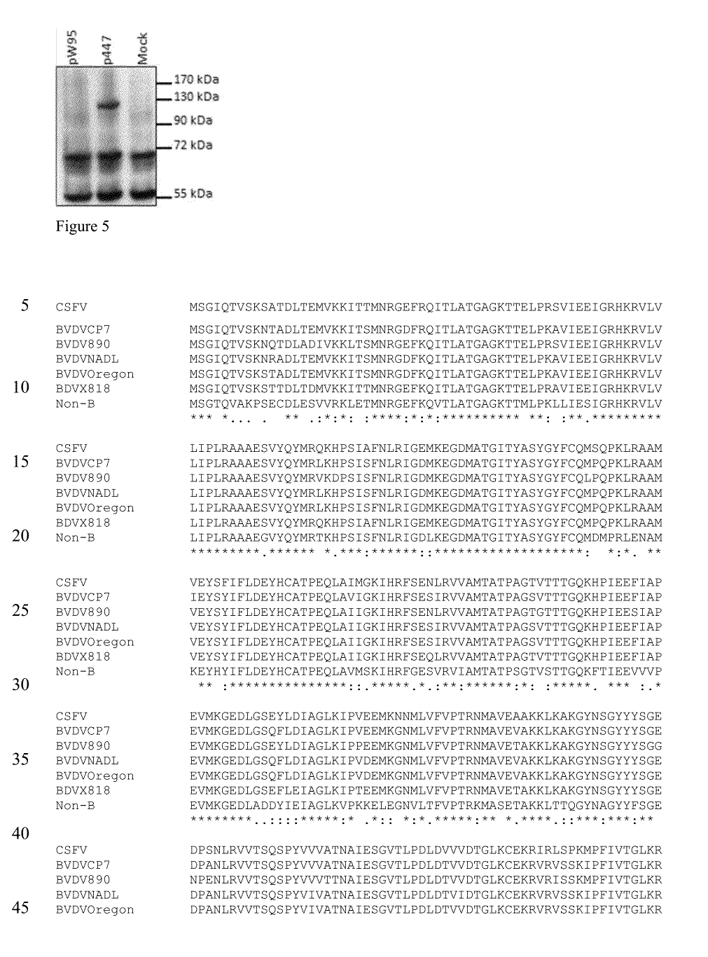

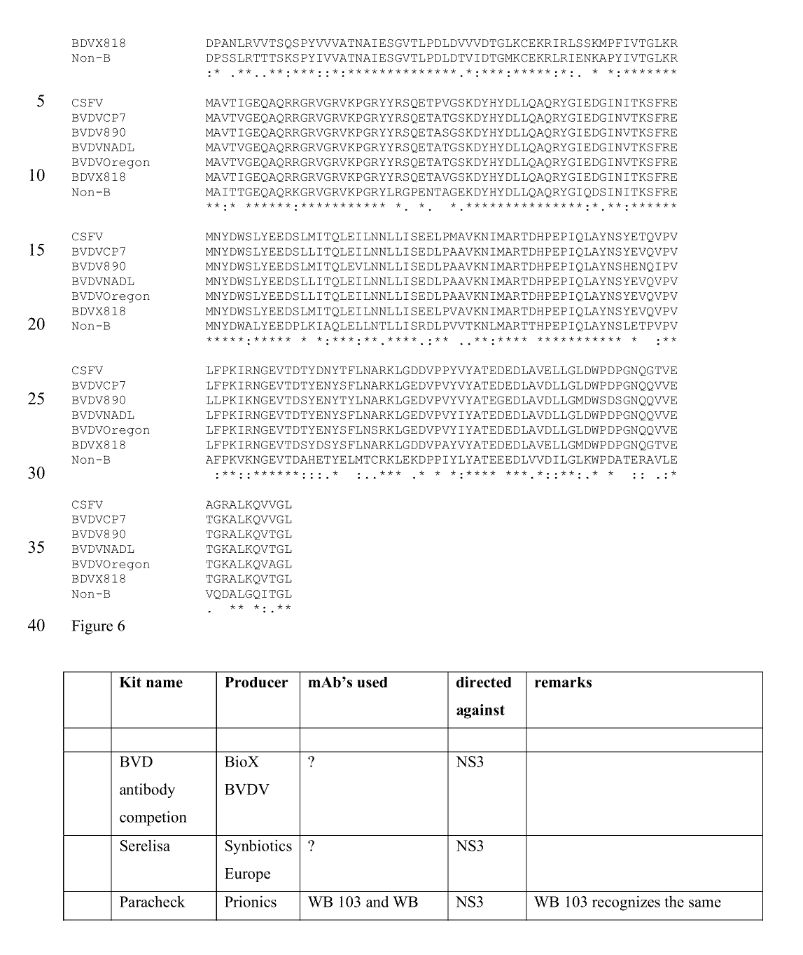

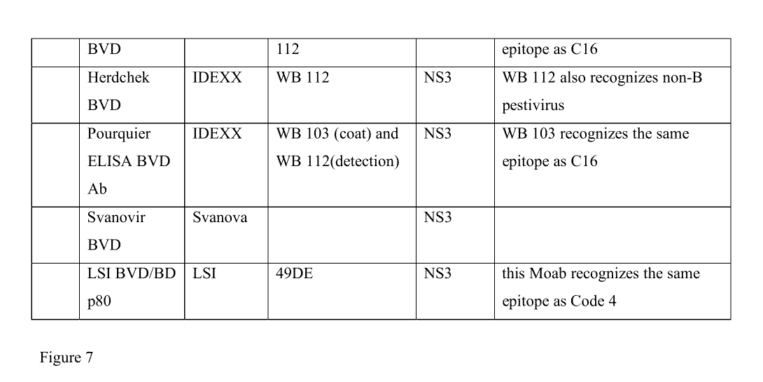

MAb 14E7 was raised against NS3 D3 spanning 180 amino acids. To map the epitope a consecutive C-terminal truncation of about 16 codons was carried out based on plasmid pL200. MAb 14E7 lost its reactivity with deletion of amino acids ₂₁₈₅LLISEDLPAAVKNIMA₂₂₀₀ indicating that the linear epitope is located within or around this stretch of amino acids. Alignment with other pestivirus isolates indicated four amino acid changes of Non-BVDV/CSFV/BDV pestivirus NS3 D3 within the otherwise well conserved (14/16 aa) peptide sequence. Using primers CST482 and CST483, the corresponding sequence was changed to “LLISRDLPVVTKNIMA” in the full-length clone pW95 (mutated sequences underlined, also see FIG. 4). Virus (VpW95) rescued from transfection of pW95 was viable and it replicated undistinguishable from CSFV wt. The mutated NS3, present in VpW95 infected cells was not detected by mAbl4E7 (FIG. 5). This also applied to bacterially expressed NS3 carrying the same mutations within the mAb 14E7 epitope.

### LITERATURE

Bathia, S. et al., Res. In Vet. Science 85: 339-45 (2008).
Beaudeau, F. et al., Vet Microbiol. 80: 329-337 (2001).
Behrens et al., J. Virol., 72: 2364-272, (1998).
Cay B., Chappuis G., Coulibaly C., Dinter Z., Edwards S., Greiser-Wilke I., Gunn M., Have P., Hess G., Juntti N., et al., Vet Microbiol. 20(2): 123-129 (1989).
Chimenzo Zoth, S. and Taboga, 0., J. of Virol. Methods 138: 99-108 (2006).
Chou, P. Y. and Fasman, G. D., Advances in Enzymology 47: 45-148 (1987).
Clackson, T. & Wells, J. A. in Trends Biotechn. 12: 173-183(1994).
Colijn, E. O. et al, Vet. Microbiology 59: 15-25 (1997)
Collett, M. S., Comp. Immunol, Microbiol. And infect. Diseases 15: 145-154 (1992).
Corapi W. V., Donis R. O., Dubovi E. J. J., Virol. 62(8): 2823-2827 (1988).
Corapi, W. V. et al., Am. J. Vet. Res. 51: 1388-1393 (1990).
Cortese, R. et al., in Trends Biotechn. 12: 262-267 (1994).
Deregt, D., et al., Can. J. Vet. Res. 54: 343-348 (1990).
Deregt, D. et al., Vet. Microbiol. 108: 13-22 (2005).
Dyson, M., Expression systems, edited by Michael Dyson and Yves Durocher, Scion Publishing Ltd, ISBN 9781904842439 (edition 2007).
Edwards S., Sands J. J., Harkness J. W., Arch Virol. 102(3-4): 197-206 (1988).
Edwards S., Moennig V., Wensvoort G., Vet Microbiol. 29(2): 101-108 (1991).
Gellissen, G., Production of Recombinant Proteins: Novel Microbial and Eukaryotic Expression Systems, Publisher: Wiley-VCH, ISBN: 3527310363 (edition 2005).
Geysen M., et al., Proc. Natl Acad. Sci. 81: 3998-4002 (1984).
Geysen M., et al., J. Imm. Meth. 102: 259-274 (1987).
Ghahroudi, M. A. et al., FEBS Letters 414: 512-526 (1997).
Hopp, T. P. and Woods, K. R., Proc. Natl. Acad. Sci. 78: 38248-3828 (1981).
Jian Xu et al., J. of Virol., 71: 5312-5322 (1997) Kasza L., Shadduck J., Christofinis G., Res. Vet. Sci. 13: 46-51 (1972).
Kohler, G and Milstein, C., Nature 256: 495-497 (1975).
Kramps, J. A. et al., Vet. Microbiol. 64: 135-144 (1999).
Lai, M. M. C., et al, J. Virol, 74: 6339-6347 (2000).
Lamp, B. et al., J. Virol. 85: 3607-3620 (2011).
Little, M. et al., Biotechn. Adv. 12: 539-555 (1994).
Macpherson I. A., Stoker M. G. P., Virology 16: 147-151 (1962).
Makoschey, B. et al., Vaccine 25: 6140-6145 (2007).
Marks, J. D. et al., in J. Biol. Chem. 267: 16007-16010(1992).
Maurer, R. et al., Vaccine, 23: 3318-3328 (2005).
Mayer, D. et al., Vaccine 22: 317-328 (2004).
Meyer, B. and Peters, Th., in Angewandte Chemie International Edition, Volume 42, Issue 8, pages 864-890, Feb. 24, 2003. © 2002 WILEY-VCH Verlag GmbH & Co. KGaA, Weinheim.
Meyers, G. et al., J. virol. 70: 1588-1595 (1996)^{a}.
Meyers, G. et al., J. virol. 70: 8606-8613 (1996)^{b}.
Meyers, G. et al., J. Virol. 73: 10224-10235 (1999).
Ming Xiao et al., J. gen. Virol. 89: 994-999 (2008).
Moenning, V., Bolin, S. R., Coulibay, C. O. Z., Deut. Tierarztl. Woch. 94: 572-576 (1987).
Moormann et al., J. of Virology 70: 763-770 (1996).
Moser et al., J. Virol. 73: 7787-7794 (1999).
Muyldermans, S. and Lauwereys, M., Journ. Molec. Recogn. 12: 131-140 (1999).
Paton D. J., Ibata G., Edwards S., Wensvoort G. J., Virol Methods. 31: 315-24 (1991).
Peters W., Greiser-Wilke I., Moennig V., Liess B., Vet Microbiol. 12: 195-200 (1986).
Riedel C., Lamp B., Heimann M., Rümenapf T., J Virol. 84: 11523-31 (2010).
Riedel, C. et al, PLoS Pathog. 2012; 8(3): e1002598. doi: 10.1371/journal.ppat.1002598. Epub 2012 Mar. 22.
Risatti, G. R. et al., Virology 364: 371-382 (2007).
Robiolo B. et al., J. Virol. Methods. 166(1-2): 21-27 (2010)
Schägger H., von Jagow G. Anal Biochem. 166(2): 368-79 (1987).
Tackett, A. J. et al., Nucleic Acids Res. 29: 565-572 (2001).
Tautz, N. et al., Virology 273: 351-363 (2000).
Tratschin, J., et al., Journ. Virol. 72: 7681-7684 (1998).
Trepe, K., Applied Microbiology and Biotechnology: 211-222 (2006).
Wei Cheng et al., P.N.A.S. 104: 13954-13959 (2007).
Wensvoort G. et al., Vet. Microbiol. 17(2): 129-140 (1988)
Widjojoatmodjo et al., J. Virol. 74: 2973-80 (2000).
Winter, G. et al., in Annu. Rev. Immunol. 12: 433-455 (1994)
Yingming Zhao and Chalt, B. T., Anal. Chemistry 66: 3723-3726 (1994).

1-18. (canceled)

19. A replication-competent virus comprising a modification in an epitope of a viral non-structural protein 3 (NS3);
wherein the epitope is located in a helicase domain in the NS3;
wherein as a result of said modification, the epitope is no longer reactive with a monoclonal antibody against that epitope in the NS3 of the corresponding wild-type virus; and
wherein said replication-competent virus is selected from the group consisting of Bovine viral diarrhoea virus (BVDV), Classical Swine Fever virus (CSFV), atypical pestivirus and Ovine Border Disease viruses (BDV).

20. The replication-competent virus of claim 19, wherein said helicase domain is selected from the group consisting of helicase domain 1, 2 or 3.

21. The replication-competent virus of claim 20, wherein said helicase domain is selected from the group consisting of CSFV Alfort Tuebingen, located between amino acid position 1782 and position 2272, BVDV-1 CP7, located between amino acid position 1791 and position 2281, BVDV-1 NCP7, located between amino acid position 1782 and position 2272, BVDV-1 NADL, located between amino acid position 1872 and position 2362, BVDV-1 Oregon C24V, located between amino acid position 1782 and position 2272, BVDV-2 890, located between amino acid position 1856 and position 2346, and BDV X818, located between amino acid position 1779 and position 2269.

22. The replication-competent virus of claim 19, wherein said monoclonal antibody is selected from the group consisting of mAb BVD/C16-INT, mAb 8.12.7αNS3h, Code4 and mAb 14E7αNS3h, GL3h6.

23. The replication-competent virus of claim 19 that is inactivated.

24. A vaccine comprising the replication-competent virus of claim 23 and a pharmaceutically acceptable carrier.

25. A vaccine comprising the replication-competent virus of claim 19 and a pharmaceutically acceptable carrier.

26. The vaccine of claim 25, wherein said replication-competent virus carries an attenuating mutation in the E^{rns} or the N^{pro} gene.

27. The vaccine of claim 25 that further comprises a component selected from the group consisting of an additional immunogen of a virus that is pathogenic to the animal to be vaccinated, an antibody against said additional immunogen of said virus, genetic information encoding said additional immunogen of said virus, an additional immunogen of a micro-organism that is pathogenic to the animal to be vaccinated, an antibody against said additional immunogen of said micro-organism, and genetic information encoding said additional immunogen of said micro-organism.

28. The vaccine of claim 27, wherein said virus pathogenic to the animal to be vaccinated is selected from the group consisting of Bovine Rotavirus, epizootic Haemorrhagic Disease virus, Rift Valley Fever virus, Bovine ephemeral fever virus, Bovine Herpesvirus, Parainfluenza Type 3 virus, Bovine Paramyxovirus, Bluetongue virus, Orthobunya virus, Foot and Mouth Disease virus, and Bovine Respiratory Syncytial Virus; and
wherein said micro-organism pathogenic to the animal to be vaccinated is selected from the group consisting of Mannheimia haemolytica and Pasteurella multocida.

29. The vaccine of claim 27, wherein said virus pathogenic to the animal to be vaccinated is selected from the group consisting of African Swine Fever virus, Nipah virus, Porcine Circovirus, Porcine Torque Teno virus, Pseudorabies virus, Porcine influenza virus, Porcine parvo virus, Porcine respiratory and Reproductive syndrome virus (PRRS), Porcine Epidemic Diarrheal virus (PEDV), Foot and Mouth disease virus, Transmissible gastro-enteritis virus, and Rotavirus; and
wherein said micro-organism pathogenic to the animal to be vaccinated is selected from the group consisting of Brachyspira hyodysenteriae, Escherichia coli, Erysipelo rhusiopathiae, Bordetella bronchiseptica, Salmonella cholerasuis, Haemophilus parasuis, Pasteurella multocida, Streptococcus suis, Mycoplasma hyopneumoniae and Actinobacillus pleuropneumoniae.

30. The vaccine of claim 27, wherein said virus pathogenic to the animal to be vaccinated is selected from the group consisting of Foot and Mouth disease virus, Rift Valley Fever virus, Orthobunya virus, Louping Ill, Peste des petits Ruminants, Nairobi sheep disease virus, Bluetongue virus, Caprine Arthritis Encephalitis Virus (CAEV), and Ovine Herpesvirus; and
wherein said micro-organism pathogenic to the animal to be vaccinated is selected from the group consisting of E. coli, Chlamydia psittaci, Clostridium perfringens, Clostridium septicum, Clostridium titani, Clostridium novyi, Clostridium chauvoei, Toxoplasma gondii, Pasteurella haemolytica and Pasteurella trehalosi.

31. A diagnostic test for distinguishing mammals vaccinated with the vaccine of claim 25 from mammals that have been infected with a wild-type BVDV, CSFV, atypical pestivirus or BDV, wherein said diagnostic test comprises an NS3 epitope of a wild-type BVDV, CSFV, atypical pestivirus or BDV.

32. A diagnostic test for distinguishing mammals vaccinated with the vaccine of claim 25 from mammals that have been infected with a wild-type BVDV, CSFV, atypical pestivirus or BDV, wherein said diagnostic test comprises an antibody against an NS3 epitope of a wild-type BVDV, CSFV, atypical pestivirus or BDV.

33. A diagnostic test for distinguishing mammals vaccinated with the vaccine of claim 25 from mammals that have been infected with a wild-type BVDV, CSFV, atypical pestivirus or BDV, wherein said diagnostic test comprises an epitope of a helicase domain in the non-structural protein NS3, said epitope having a modification as a result of which the epitope is no longer reactive with a monoclonal antibody against that epitope in a wild-type BVDV, CSFV, atypical pestivirus or BDV.

34. A diagnostic test for distinguishing mammals vaccinated with the vaccine of claim 25 from mammals that have been infected with a wild-type BVDV, CSFV, atypical pestivirus or BDV, wherein said diagnostic test comprises an antibody against an epitope of a helicase domain in the non-structural protein NS3, wherein said epitope has a modification as a result of which the epitope is no longer reactive with a monoclonal antibody against that epitope in a wild-type BVDV, CSFV, atypical pestivirus or BDV.

35. A method for distinguishing a mammal vaccinated with the vaccine of claim 25 from a mammal that has been infected with a wild-type BVDV, CSFV, atypical pestivirus or BDV comprising collecting a test sample from a mammal that contains an antibody against BVDV, CSFV, atypical pestivirus or BDV; and
ascertaining whether the antibodies are reactive with the wild-type virus or are reactive with the replication-competent virus comprising a modification in the epitope of the viral non-structural protein 3 (NS3).

36. The method of claim 35, wherein said ascertaining is performed by ELISA.

37. The method of claim 36, wherein the wild-type version of the epitope in the helicase domain of the non-structural protein NS3 is coated.

38. The method of claim 36, wherein the antibody reactive with the wild-type form of the epitope in a helicase domain of the non-structural protein NS3 is coated.


SEQUENCE LISTING (95 sequences)

SEQ ID NO: 1 - DNA, 2049 bp, Pestivirus
  CDS (1)..(2049)
gggccagccgtctgcaagaagataatacaccatgagaaatgcaggccttcaataccagacaaactcatggcattcttcgggattatgcctaggggagttacaccaagagcccctacacggttccctgtgtccttgctgaagataagacggggttttgagaccggctgggcctacacacaccctggaggggtaagtagtgtgatgcatgtcaccgctgggtcggatatatatgtcaatgactcaatagggaggacaaaaatccagtgccaagacaaaaacactacaacagatgagtgtgaatatggtgtgaaaacagactcagggtgctctgatggagctcggtgctatgtcatcaaccctgaagcaaccaacatagcagggaccaagggggccatggtacacctgaggaaagctggaggagagttcaactgcgtgactgcccagggtacccccgccttctataatctaaagaacttaaaaggatggtcaggcctgcctatctttgaagctgccacaggaagagtggtaggaagggtaaaagcaggaaaaaacactgacaatgctccaacaaccattatgtcagggacgcaagtggcaaaaccatcagagtgtgacctagaatcagtggtgaggaaactagagacaatgaacagaggggaattcaaacaagtgactctggctacaggcgcaggaaagacaaccatgctaccaaagctgttaatagaatccataggcaggcataagagagtgttagtactgatcccgttgagagctgcagcggagggggtgtaccagtacatgagaaccaaacacccaagcatatctttcaacttgaggataggggatctgaaagaaggtgacatggcaactgggatcacctatgcctcttatgggtacttctgccaaatggacatgcctagactggagaatgcaatgaaggaataccactatattttcttggatgaatatcactgtgccacaccagaacagttggcagtgatgtcaaaaatacataggttcggtgaatcagttagggtaatagccatgaccgccacgccatccgggactgtgagcacaacagggcagaaattcacaattgaggaggtggtagtacctgaagtgatgaagggggaggaccttgctgatgattacatcgaaatagcagggttgaaggtgccaaagaaagagttagagggtaacgtactgacttttgtgcctacaaggaagatggcatcggaaacagcaaaaaaattaaccacacagggatacaatgctggatactacttcagtggagaagatccatcatccctgcggacaactacttctaagtcaccatatatagtagttgcaaccaatgccattgaatccggggtaaccttaccggaccttgatacagtaatagatacaggcatgaagtgtgaaaagagactaagaatcgaaaacaaagctccctacatcgtaacaggactgaaaagaatggctataacaacgggggagcaagctcaaagaaaaggtagggtaggcagggttaaacctgggaggtacttgagaggacctgaaaacactgcaggtgaaaaggactatcactatgaccttttacaggcacagaggtacggcatccaagactcaataaacatcaccaagtctttcagggagatgaactatgattgggcattatatgaggaagacccgttaaagattgcccaattagagttgctaaacacactcctgatctcaagggatctgccagtagtaacaaaaaatctgatggcccgcacaacacatcccgaacctatacaattggcttacaatagtttagaaacccctgtaccggtggcattcccaaaagtgaaaaatggagaagtcactgacgcacatgaaacttacgagttgatgacctgtaggaagcttgagaaagacccccctatatacctgtatgcaacagaagaagaagatctcgtagtggacatactgggattgaaatggccagacgccacagagagggctgtcttggaagtgcaagacgccctgggccagatcacaggttta

SEQ ID NO: 2 - protein, 683 aa, Pestivirus
GPAVCKKIIHHEKCRPSIPDKLMAFFGIMPRGVTPRAPTRFPVSLLKIRRGFETGWAYTHPGGVSSVMHVTAGSDIYVNDSIGRTKIQCQDKNTTTDECEYGVKTDSGCSDGARCYVINPEATNIAGTKGAMVHLRKAGGEFNCVTAQGTPAFYNLKNLKGWSGLPIFEAATGRVVGRVKAGKNTDNAPTTIMSGTQVAKPSECDLESVVRKLETMNRGEFKQVTLATGAGKTTMLPKLLIESIGRHKRVLVLIPLRAAAEGVYQYMRTKHPSISFNLRIGDLKEGDMATGITYASYGYFCQMDMPRLENAMKEYHYIFLDEYHCATPEQLAVMSKIHRFGESVRVIAMTATPSGTVSTTGQKFTIEEVVVPEVMKGEDLADDYIEIAGLKVPKKELEGNVLTFVPTRKMASETAKKLTTQGYNAGYYFSGEDPSSLRTTTSKSPYIVVATNAIESGVTLPDLDTVIDTGMKCEKRLRIENKAPYIVTGLKRMAITTGEQAQRKGRVGRVKPGRYLRGPENTAGEKDYHYDLLQAQRYGIQDSINITKSFREMNYDWALYEEDPLKIAQLELLNTLLISRDLPVVTKNLMARTTHPEPIQLAYNSLETPVPVAFPKVKNGEVTDAHETYELMTCRKLEKDPPIYLYATEEEDLVVDILGLKWPDATERAVLEVQDALGQITGL

SEQ ID NO: 3 - DNA, 24 bp, Pestivirus
caggaaacagcaaccgggtcaaag

SEQ ID NO: 4 - DNA, 24 bp, Pestivirus
gctagccatatgtatatctccttc

SEQ ID NO: 5 - DNA, 22 bp, Pestivirus
caccaccaccaccaccaccatc

SEQ ID NO: 6 - DNA, 21 bp, Pestivirus
tgttgtggttactgaccctgc

SEQ ID NO: 7 - DNA, 22 bp, Pestivirus
gggcaaaaacacccaatagaag

SEQ ID NO: 8 - DNA, 24 bp, Pestivirus
gctagccatatgtatatctccttc

SEQ ID NO: 9 - DNA, 22 bp, Pestivirus
caccaccaccaccaccaccatc

SEQ ID NO: 10 - DNA, 25 bp, Pestivirus
acttctataatacctaccgggtttc

SEQ ID NO: 11 - DNA, 20 bp, Pestivirus
caagaaacacctgtcggctc

SEQ ID NO: 12 - DNA, 23 bp, Pestivirus
agtggttgttactgtgcctgccg

SEQ ID NO: 13 - DNA, 21 bp, Pestivirus
gggcagaaattcacaattgag

SEQ ID NO: 14 - DNA, 20 bp, Pestivirus
tcctctcaagtacctcccag

SEQ ID NO: 15 - DNA, 27 bp, Pestivirus
gcaaagaaattgaaggccaaaggatac

SEQ ID NO: 16 - DNA, 22 bp, Pestivirus
cgcctctaccgccatgttcctg

SEQ ID NO: 17 - DNA, 27 bp, Pestivirus
gcaaaaaaattaaccacacagggatac

SEQ ID NO: 18 - DNA, 23 bp, Pestivirus
tgtttccgatgccatcttccttg

SEQ ID NO: 19 - DNA, 27 bp, Pestivirus
gcaaaaaaattaaccacacagggatac

SEQ ID NO: 20 - DNA, 23 bp, Pestivirus
tgtttccgatgccatcttccttg

SEQ ID NO: 21 - DNA, 27 bp, Pestivirus
gcaaagaaattgaaggccaaaggatac

SEQ ID NO: 22 - DNA, 22 bp, Pestivirus
cgcctctaccgccatgttcctg

SEQ ID NO: 23 - DNA, 24 bp, Pestivirus
ttcgatgtaatcatcagcaaggtc

SEQ ID NO: 24 - DNA, 27 bp, Pestivirus
gcaaaaaaattaaccacacagggatac

SEQ ID NO: 25 - DNA, 24 bp, Pestivirus
attgccggactgaagataccagta

SEQ ID NO: 26 - DNA, 23 bp, Pestivirus
cttagaaggcacagtcgaggctg

SEQ ID NO: 27 - DNA, 24 bp, Pestivirus
accctctaactctttctttggcac

SEQ ID NO: 28 - DNA, 27 bp, Pestivirus
gcaaaaaaattaaccacacagggatac

SEQ ID NO: 29 - DNA, 23 bp, Pestivirus
aacatgctagtttttgtgcccac

SEQ ID NO: 30 - DNA, 23 bp, Pestivirus
cttagaaggcacagtcgaggctg

SEQ ID NO: 31 - DNA, 26 bp, Pestivirus
ttcaggtactaccacctcctcaattg

SEQ ID NO: 32 - DNA, 27 bp, Pestivirus
gcaaaaaaattaaccacacagggatac

SEQ ID NO: 33 - DNA, 22 bp, Pestivirus
gtgatgaaaggagaagacttgg

SEQ ID NO: 34 - DNA, 23 bp, Pestivirus
cttagaaggcacagtcgaggctg

SEQ ID NO: 35 - DNA, 29 bp, Pestivirus
gagaataacatgctagtttttgtgcccac

SEQ ID NO: 36 - DNA, 30 bp, Pestivirus
cagctcctctactggtatcttcagtccggc

SEQ ID NO: 37 - DNA, 25 bp, Pestivirus
ctagaaacacctgtcggctctaaag

SEQ ID NO: 38 - DNA, 25 bp, Pestivirus
actcctgtagtatcttccaggcttc

SEQ ID NO: 39 - DNA, 25 bp, Pestivirus
ctagaaacacctgtcggctctaaag

SEQ ID NO: 40 - DNA, 25 bp, Pestivirus
actcctgtagtatcttccaggcttc

SEQ ID NO: 41 - DNA, 28 bp, Pestivirus
cacaataatctgtccaaaatagttgaac

SEQ ID NO: 42 - DNA, 25 bp, Pestivirus
gttccagctcttgtatgtataagtc

SEQ ID NO: 43 - DNA, 25 bp, Pestivirus
cagatctcgtgatatcaacaggttg

SEQ ID NO: 44 - DNA, 27 bp, Pestivirus
ccggtggtaacaaaaaatataatggcc

SEQ ID NO: 45 - DNA, 25 bp, Pestivirus
gctcctgtagtatcttccaggcttc

SEQ ID NO: 46 - DNA, 20 bp, Pestivirus
caagaaacacctgtcggctc

SEQ ID NO: 47 - DNA, 25 bp, Pestivirus
cctgaaaacactgcaggtgaaaagg

SEQ ID NO: 48 - DNA, 22 bp, Pestivirus
ggttatgctagttattgctcag

SEQ ID NO: 49 - DNA, 25 bp, Pestivirus
cactgggcagaaacaccctatagag

SEQ ID NO: 50 - DNA, 23 bp, Pestivirus
agtggttgttactgtgcctgccg

SEQ ID NO: 51 - DNA, 19 bp, Pestivirus
gtgctcacagtcccggatg

SEQ ID NO: 52 - DNA, 19 bp, Pestivirus
ggaattgtgagcggataac

SEQ ID NO: 53 - DNA, 20 bp, Pestivirus
caagaaacacctgtcggctc

SEQ ID NO: 54 - DNA, 23 bp, Pestivirus
agtggttgttactgtgcctgccg

SEQ ID NO: 55 - DNA, 21 bp, Pestivirus
gggcagaaattcacaattgag

SEQ ID NO: 56 - DNA, 20 bp, Pestivirus
tcctctcaagtacctcccag

SEQ ID NO: 57 - DNA, 25 bp, Pestivirus
aaacatatgagtgggatacaaacgg

SEQ ID NO: 58 - DNA, 27 bp, Pestivirus
aaactcgagttatagaccaaccacctg

SEQ ID NO: 59 - DNA, 21 bp, Pestivirus
caattgtataggttcgggatg

SEQ ID NO: 60 - DNA, 24 bp, Pestivirus
taactcgagcaccaccaccaccac

SEQ ID NO: 61 - DNA, 22 bp, Pestivirus
gcgtataacagctacgagacac

SEQ ID NO: 62 - DNA, 24 bp, Pestivirus
gtggtggtggtggtgctcgagtta

SEQ ID NO: 63 - DNA, 21 bp, Pestivirus
gagttgaattggttctgggtg

SEQ ID NO: 64 - DNA, 24 bp, Pestivirus
taactcgagcaccaccaccaccac

SEQ ID NO: 65 - DNA, 23 bp, Pestivirus
gcttacaatagtttagaaacccc

SEQ ID NO: 66 - DNA, 24 bp, Pestivirus
gtggtggtggtggtgctcgagtta

SEQ ID NO: 67 - DNA, 24 bp, Pestivirus
cacgtaggggggtacgtcatctcc

SEQ ID NO: 68 - DNA, 24 bp, Pestivirus
taactcgagcaccaccaccaccac

SEQ ID NO: 69 - DNA, 25 bp, Pestivirus
tatgcaacagaagaagaagatctcg

SEQ ID NO: 70 - DNA, 24 bp, Pestivirus
gtggtggtggtggtgctcgagtta

SEQ ID NO: 71 - DNA, 24 bp, Pestivirus
taactcgagcaccaccaccaccac

SEQ ID NO: 72 - DNA, 20 bp, Pestivirus
ctgccagcttccacggtgcc

SEQ ID NO: 73 - DNA, 24 bp, Pestivirus
taactcgagcaccaccaccaccac

SEQ ID NO: 74 - DNA, 24 bp, Pestivirus
cacgtaggggggtacgtcatctcc

SEQ ID NO: 75 - DNA, 24 bp, Pestivirus
taactcgagcaccaccaccaccac

SEQ ID NO: 76 - DNA, 22 bp, Pestivirus
tcttatttttgggaataatacc

SEQ ID NO: 77 - DNA, 24 bp, Pestivirus
taactcgagcaccaccaccaccac

SEQ ID NO: 78 - DNA, 21 bp, Pestivirus
ctgccatcggcagctcttctg

SEQ ID NO: 79 - DNA, 24 bp, Pestivirus
taactcgagcaccaccaccaccac

SEQ ID NO: 80 - DNA, 21 bp, Pestivirus
cgtagttcatctctctgaagg

SEQ ID NO: 81 - DNA, 20 bp, Pestivirus
caagaaacacctgtcggctc

SEQ ID NO: 82 - DNA, 21 bp, Pestivirus
cgtagttcatctctctgaagg

SEQ ID NO: 83 - DNA, 27 bp, Pestivirus
atgggtggtggccatggtattatcatc

SEQ ID NO: 84 - DNA, 25 bp, Pestivirus
cactgggcagaaacaccctatagag

SEQ ID NO: 85 - DNA, 24 bp, Pestivirus
taactcgagcaccaccaccaccac

SEQ ID NO: 86 - DNA, 25 bp, Pestivirus
actcctgtagtatcttccaggcttc

SEQ ID NO: 87 - DNA, 22 bp, Pestivirus
agtggttgttactgtgcctgcc

SEQ ID NO: 88 - DNA, 20 bp, Pestivirus
caagaaacacctgtcggctc

SEQ ID NO: 89 - protein, 491 aa, Pestivirus
MSGIQTVSKSATDLTEMVKKITTMNRGEFRQITLATGAGKTTELPRSVIEEIGRHKRVLVLIPLRAAAESVYQYMRQKHPSIAFNLRIGEMKEGDMATGITYASYGYFCQMSQPKLRAAMVEYSFIFLDEYHCATPEQLAIMGKIHRFSENLRVVAMTATPAGTVTTTGQKHPIEEFIAPEVMKGEDLGSEYLDIAGLKIPVEEMKNNMLVFVPTRNMAVEAAKKLKAKGYNSGYYYSGEDPSNLRVVTSQSPYVVVATNAIESGVTLPDLDVVVDTGLKCEKRIRLSPKMPFIVTGLKRMAVTIGEQAQRRGRVGRVKPGRYYRSQETPVGSKDYHYDLLQAQRYGIEDGINITKSFREMNYDWSLYEEDSLMITQLEILNNLLISEELPMAVKNIMARTDHPEPIQLAYNSYETQVPVLFPKIRNGEVTDTYDNYTFLNARKLGDDVPPYVYATEDEDLAVELLGLDWPDPGNQGTVEAGRALKQVVGL

SEQ ID NO: 90 - protein, 491 aa, Pestivirus
MSGIQTVSKNTADLTEMVKKITSMNRGDFRQITLATGAGKTTELPKAVIEEIGRHKRVLVLIPLRAAAESVYQYMRLKHPSISFNLRIGDMKEGDMATGITYASYGYFCQMPQPKLRAAMIEYSYIFLDEYHCATPEQLAVIGKIHRFSESIRVVAMTATPAGSVTTTGQKHPIEEFIAPEVMKGEDLGSQFLDIAGLKIPVEEMKGNMLVFVPTRNMAVEVAKKLKAKGYNSGYYYSGEDPANLRVVTSQSPYVVVATNAIESGVTLPDLDTVVDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNARKLGEDVPVYVYATEDEDLAVDLLGLDWPDPGNQQVVETGKALKQVVGL

SEQ ID NO: 91 - protein, 491 aa, Pestivirus
MSGIQTVSKNQTDLADIVKKLTSMNRGEFKQITLATGAGKTTELPRSVIEEIGRHKRVLVLIPLRAAAESVYQYMRVKDPSISFNLRIGDMKEGDMATGITYASYGYFCQLPQPKLRAAMVEYSYIFLDEYHCATPEQLAIIGKIHRFSENLRVVAMTATPAGTGTTTGQKHPIEESIAPEVMKGEDLGSEYLDIAGLKIPPEEMKGNMLVFVPTRNMAVETAKKLKAKGYNSGYYYSGGNPENLRVVTSQSPYVVVTTNAIESGVTLPDLDTVVDTGLKCEKRVRISSKMPFIVTGLKRMAVTIGEQAQRRGRVGRVKPGRYYRSQETASGSKDYHYDLLQAQRYGIEDGINVTKSFREMNYDWSLYEEDSLMITQLEVLNNLLISEDLPAAVKNIMARTDHPEPIQLAYNSHENQIPVLLPKIKNGEVTDSYENYTYLNARKLGEDVPVYVYATEGEDLAVDLLGMDWSDSGNQQVVETGRALKQVTGL

SEQ ID NO: 92 - protein, 491 aa, Pestivirus
MSGIQTVSKNRADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPEVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVIDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNARKLGEDVPVYIYATEDEDLAVDLLGLDWPDPGNQQVVETGKALKQVTGL

SEQ ID NO: 93 - protein, 491 aa, Pestivirus
MSGIQTVSKSTADLTEMVKKITSMNRGDFKQITLATGAGKTTELPKAVIEEIGRHKRVLVLIPLRAAAESVYQYMRLKHPSISFNLRIGDMKEGDMATGITYASYGYFCQMPQPKLRAAMVEYSYIFLDEYHCATPEQLAIIGKIHRFSESIRVVAMTATPAGSVTTTGQKHPIEEFIAPEVMKGEDLGSQFLDIAGLKIPVDEMKGNMLVFVPTRNMAVEVAKKLKAKGYNSGYYYSGEDPANLRVVTSQSPYVIVATNAIESGVTLPDLDTVVDTGLKCEKRVRVSSKIPFIVTGLKRMAVTVGEQAQRRGRVGRVKPGRYYRSQETATGSKDYHYDLLQAQRYGIEDGINVTKSFREMNYDWSLYEEDSLLITQLEILNNLLISEDLPAAVKNIMARTDHPEPIQLAYNSYEVQVPVLFPKIRNGEVTDTYENYSFLNSRKLGEDVPVYIYATEDEDLAVDLLGLDWPDPGNQQVVETGKALKQVAGL

SEQ ID NO: 94 - protein, 491 aa, Pestivirus
MSGIQTVSKSTTDLTDMVKKITTMNRGEFKQITLATGAGKTTELPRAVIEEIGRHKRVLVLIPLRAAAESVYQYMRQKHPSIAFNLRIGEMKEGDMATGITYASYGYFCQMPQPKLRAAMVEYSYIFLDEYHCATPEQLAIIGKIHRFSEQLRVVAMTATPAGTVTTTGQKHPIEEFIAPEVMKGEDLGSEFLEIAGLKIPTEEMKGNMLVFVPTRNMAVETAKKLKAKGYNSGYYYSGEDPANLRVVTSQSPYVVVATNAIESGVTLPDLDVVVDTGLKCEKRIRLSSKMPFIVTGLKRMAVTIGEQAQRRGRVGRVKPGRYYRSQETAVGSKDYHYDLLQAQRYGIEDGINITKSFREMNYDWSLYEEDSLMITQLEILNNLLISEELPVAVKNIMARTDHPEPIQLAYNSYEVQVPVLFPKIRNGEVTDSYDSYSFLNARKLGDDVPAYVYATEDEDLAVELLGMDWPDPGNQGTVETGRALKQVTGL

SEQ ID NO: 95 - protein, 491 aa, Pestivirus
MSGTQVAKPSECDLESVVRKLETMNRGEFKQVTLATGAGKTTMLPKLLIESIGRHKRVLVLIPLRAAAEGVYQYMRTKHPSISFNLRIGDLKEGDMATGITYASYGYFCQMDMPRLENAMKEYHYIFLDEYHCATPEQLAVMSKIHRFGESVRVIAMTATPSGTVSTTGQKFTIEEVVVPEVMKGEDLADDYIEIAGLKVPKKELEGNVLTFVPTRKMASETAKKLTTQGYNAGYYFSGEDPSSLRTTTSKSPYIVVATNAIESGVTLPDLDTVIDTGMKCEKRLRIENKAPYIVTGLKRMAITTGEQAQRKGRVGRVKPGRYLRGPENTAGEKDYHYDLLQAQRYGIQDSINITKSFREMNYDWALYEEDPLKIAQLELLNTLLISRDLPVVTKNLMARTTHPEPIQLAYNSLETPVPVAFPKVKNGEVTDAHETYELMTCRKLEKDPPIYLYATEEEDLVVDILGLKWPDATERAVLEVQDALGQITGL